SEQUENCE LISTING (6 sequences)

SEQ ID NO: 1 - DNA, 15 bp, Artificial Sequence
  Description of Artificial Sequence Synthetic oligonucleotide
agatcaccatcccta

SEQ ID NO: 2 - DNA, 11 bp, Artificial Sequence
  Description of Artificial Sequence Synthetic oligonucleotide
agatcaccacc

SEQ ID NO: 3 - DNA, 15 bp, Artificial Sequence
  Description of Artificial Sequence Synthetic oligonucleotide
agatgacgaccccta

SEQ ID NO: 4 - DNA, 11 bp, Artificial Sequence
  Description of Artificial Sequence Synthetic oligonucleotide
gacgacaccgc

SEQ ID NO: 5 - DNA, 15 bp, Artificial Sequence
  Description of Artificial Sequence Synthetic oligonucleotide
agatcacgatcccgc

SEQ ID NO: 6 - DNA, 13 bp, Artificial Sequence
  Description of Artificial Sequence Synthetic oligonucleotide
agatcacgatccc

(19) United States
(12) Patent Application Publication
(10) Pub. No.: US 2022/0351804 A1
(43) Pub. Date: Nov. 3, 2022

(54) Improved Variant Caller Using Single-Cell Analysis

(71) Applicant: Mission Bio, Inc., South San Francisco (US)

(72) Inventors: Manimozhi Manivannan, South San Francisco, CA (US); Dongmyunghee Kim, South San Francisco, CA (US); Sombeet Sahu, South San Francisco, CA (US); Saurabh Gulati, South San Francisco, CA (US); Shu Wang, South San Francisco, CA (US)

(21) Appl. No.: 17/766,017

(22) PCT Filed: Oct. 2, 2020
(86) PCT No.: PCT/US2020/053971

§ 371 Date: Apr. 1, 2022

Related U.S. Application Data
(60) Provisional application No. 62/909,670, filed on Oct. 2, 2019.

(51) Int. Cl. G16B 20/00; G16B 30/10; G16B 40/20

(52) U.S. Cl. CPC G16B 20/00; G16B 30/10; G16B 40/20

(57) ABSTRACT

Described herein are improved variant calling methods including a two-step process involving 1) error correction of bases in sequence reads through a cell-specific process and 2) variant calling across cell populations using the error corrected sequence reads. Generally, the first step of error correction involves applying a first machine learned model to identify and correct bases of sequence reads. The second step of variant calling involves applying a second machine learned model to classify a base. Such improved variant calling methods can be useful for identifying variants that are implicated in biological processes, such as diseased biological processes.

### CROSS REFERENCE

This application claims the benefit of and priority to U.S. Provisional Patent Application No. 62/909,670 filed Oct. 2, 2019, the entire disclosure of which is hereby incorporated by reference in its entirety for all purposes.

### BACKGROUND

Sequencing technologies often generate sequence reads that suffer from errors that arise from PCR and sequencing errors ranging from 0.5%-2%. Variant callers that aim to call variants in cell populations often identify false positives as a result of these errors which negatively impact the accuracy of the variant caller. Conventional strategies to mitigate false positives often employ hard cutoffs; however, the implementation of these hard cutoffs eliminates a significant number of true positives, an issue often referred to as the missing data problem. Thus, there is a need for improved variant callers that can better identify false positives without sacrificing true positives.

### SUMMARY

Described herein are embodiments for improved variant calling through a two-step process involving 1) error correction of bases in sequence reads through a cell-specific process and 2) variant calling across cell populations using error corrected sequence reads. Errors in bases often arise from any of PCR errors, sequencing errors, sequencing alignment errors, or correction errors. Here, the two step process enables identification and correction of erroneous bases, thereby enabling more accurate variant calls. In various embodiments, error correction of bases involves the implementation of a first trained machine learning model, hereafter referred to as an error correction model, that is used to correct erroneous bases. Thus, the error correction model enables correction of sequence reads from individual cells. Performing error correction of bases through a cell-specific manner is advantageous in comparison to correcting sequence reads derived from bulk sequencing. For example, base errors can arise in sequence reads from a single cell and therefore, these base errors are can be corrected together for the single cell. In various embodiments, variant calling across a cell population involves the implementation of a second trained machine learning model, hereafter referred to as a variant caller model. The variant caller model analyzes the corrected sequence reads to call variants that are more likely to be true variants present in the cell population. Together, the two step process involving the implementation of the error correction model and the variant caller model achieves higher accuracy in calling true variants. This can be useful for identifying true variants that may be implicated in diseases, such as cancer.

Disclosed herein is a method for calling one or more variants of a cell population, the method comprising: obtaining a plurality of sequence reads from cells of the cell population; for a plurality of cells in the cell population, correcting sequence reads obtained from the cell, the correction comprising: identifying a base of interest of the sequence reads that differs from a reference base; applying an error correction model to analyze single cell features of the base of interest, the error correction model trained to predict a probability for the base of interest; and correcting the base of interest of the sequence reads derived from the cell; generating cell population features by aggregating corrected sequence reads across cells of the cell population, the corrected sequence reads comprising corrected bases; and applying a variant caller model to the cell population features derived from the aggregated sequence reads to identify one or more variants across the cell population.

In various embodiments, the single cell features comprise contextual sequences around the base of interest, sequencing depth of the base of interest, allele frequency of the base of interest, and allele frequency of bases in a window around the base of interest. In various embodiments, identifying a base of interest of the sequence reads comprises applying a transition matrix comprising likelihoods of transition between reference bases and mismatched bases to a probability of observing a proportion of nucleotide bases across the sequence reads for a mismatched base. In various embodiments, identifying a base of interest of the sequence reads further comprises: determining the probability of observing a proportion of nucleotide bases across the sequence reads for the mismatched base; and comparing the determined probability to a likelihood of transition from the transition matrix. In various embodiments, responsive to the determined probability being greater than the likelihood of transition, identifying the mismatched base as a base of interest. In various embodiments, the transition matrix is generated using training data comprising sequence reads derived from one or more sample populations of cells. In various embodiments, the transition matrix is generated using the plurality of sequence reads from cells of the cell population. In various embodiments, the likelihoods of transition in the transition matrix are dynamically updated as sequence reads of the one or more cells of the cell population are corrected.

In various embodiments, the error correction model is a neural network. In various embodiments, the error correction model is a deep learning neural network comprising one or more layers that learn motifs and local sequence contexts around a base of interest. In various embodiments, correcting one or more sequence reads of the plurality of sequence reads derived from the cell results comprises correcting at least 25% of bases of interest that differ from a reference base.

In various embodiments, the cell population features comprise one or more of percentage of heterozygous calls, median variant allele frequency (VAF) of heterozygous calls, median genotype quality of heterozygous calls, median read depth of heterozygous calls, percentage of homozygous calls, median VAF of homozygous calls, median genotype quality of homozygous calls, median read depth of homozygous calls, percentage of reference calls, coefficient of variation (CV) of read depth for homozygous calls, CV of read depth for heterozygous calls, CV of genotype quality of homozygous calls, CV of genotype quality of heterozygous calls, CV of VAF for homozygous calls, CV of VAF for heterozygous calls, difference between mean and median VAF for homozygous calls, difference between mean and median VAF for heterozygous calls, and amplicon GC percentage.

In various embodiments, the variant caller model predicts at least one of a heterozygous variant of interest or a homozygous variant of interest. In various embodiments, the variant caller model further predicts indeterminate variants. In various embodiments, the variant caller model is trained using training data comprising sequence reads derived from one or more cell lines and indications of known heterozygous or homozygous variants present in the one or more cell lines. In various embodiments, the application of the error correction model and the variant caller model achieves at least a two-fold increase in true variant positive predictive value at a limit of detection (LOD) of 0.5% in comparison to a conventional GTAK variant caller. In various embodiments, the application of the error correction model and the variant caller model achieves a true variant positive predictive value of at least 0.6 at a limit of detection (LOD) of 0.5%. In various embodiments, the plurality of sequence reads derived from the cell are determined through a single-cell workflow analysis. In various embodiments, the reference base is determined from a reference genome sequence. In various embodiments, the reference base is determined from one or more sequence reads obtained from a control cell.

Additionally disclosed herein is a non-transitory computer readable medium for calling one or more variants of a cell population, the non-transitory computer readable medium comprising instructions that, when executed by a processor, cause the processor to: obtain a plurality of sequence reads from cells of the cell population; for a plurality of cells in the cell population, correcting sequence reads obtained from the cell, the correction comprising: identify a base of interest of the sequence reads that differs from a reference base; apply an error correction model to analyze single cell features of the base of interest, the error correction model trained to predict a probability for the base of interest; and correct the base of interest of the sequence reads derived from the cell; generate cell population features by aggregating corrected sequence reads across cells of the cell population, the corrected sequence reads comprising corrected bases; and apply a variant caller model to the cell population features derived from the aggregated sequence reads to identify one or more variants across the cell population.

In various embodiments, the single cell features comprise contextual sequences around the base of interest, sequencing depth of the base of interest, allele frequency of the base of interest, and allele frequency of bases in a window around the base of interest. In various embodiments, the instructions that cause the processor to identify a base of interest of the sequence reads further comprises instructions that, when executed by the processor, cause the processor to apply a transition matrix comprising likelihoods of transition between reference bases and mismatched bases.

In various embodiments, the instructions that cause the processor to identify a base of interest of the sequence reads further comprises instructions that, when executed by the processor, cause the processor to: determine a probability of observing proportion of nucleotide bases across the sequence reads for a mismatched base; and compare the determined probability to a likelihood of transition from the transition matrix. In various embodiments, responsive to the determined probability being greater than the likelihood of transition, identify the mismatched base as a base of interest. In various embodiments, the transition matrix is generated using training data comprising sequence reads derived from one or more sample populations of cells. In various embodiments, the transition matrix is generated using the plurality of sequence reads from cells of the cell population. In various embodiments, the likelihoods of transition in the transition matrix are dynamically updated as sequence reads of the one or more cells of the cell population are corrected.

In various embodiments, the error correction model is a neural network. In various embodiments, the error correction model is a deep learning neural network comprising one or more layers that learn motifs and local sequence contexts around a base of interest. In various embodiments, correcting one or more sequence reads of the plurality of sequence reads derived from the cell results comprises correcting at least 25% of bases of interest that differ from a reference base. In various embodiments, the cell population features comprise one or more of percentage of heterozygous calls, median variant allele frequency (VAF) of heterozygous calls, median genotype quality of heterozygous calls, median read depth of heterozygous calls, percentage of homozygous calls, median VAF of homozygous calls, median genotype quality of homozygous calls, median read depth of homozygous calls, percentage of reference calls, coefficient of variation (CV) of read depth for homozygous calls, CV of read depth for heterozygous calls, CV of genotype quality of homozygous calls, CV of genotype quality of heterozygous calls, CV of VAF for homozygous calls, CV of VAF for heterozygous calls, difference between mean and median VAF for homozygous calls, difference between mean and median VAF for heterozygous calls, and amplicon GC percentage.

In various embodiments, the variant caller model predicts at least one of a heterozygous variant of interest or a homozygous variant of interest. In various embodiments, the variant caller model further predicts indeterminate variants. In various embodiments, the variant caller model is trained using training data comprising sequence reads derived from one or more cell lines and indications of known heterozygous or homozygous variants present in the one or more cell lines. In various embodiments, the application of the error correction model and the variant caller model achieves at least a two-fold increase in true variant positive predictive value at a limit of detection (LOD) of 0.5% in comparison to a conventional GTAK variant caller. In various embodiments, the application of the error correction model and the variant caller model achieves a true variant positive predictive value of at least 0.6 at a limit of detection (LOD) of 0.5%. In various embodiments, the plurality of sequence reads derived from the cell are determined through a single-cell workflow analysis. In various embodiments, the reference base is determined from a reference genome sequence. In various embodiments, the reference base is determined from one or more sequence reads obtained from a control cell.

Additionally disclosed herein is a system comprising: a single-cell analysis workflow device configured to generate a plurality of sequence reads for cells in a cell population; a computational device communicatively coupled to the single-cell analysis workflow device, the computational device configured to: obtain a plurality of sequence reads from cells of the cell population; for a plurality of cells in the cell population, correcting sequence reads obtained from the cell, the correction comprising: identifying a base of interest of the sequence reads that differs from a reference base; applying an error correction model to analyze single cell features of the base of interest, the error correction model trained to predict a probability for the base of interest; and correcting the base of interest of the sequence reads derived from the cell; generating cell population features by aggregating corrected sequence reads across cells of the cell population, the corrected sequence reads comprising corrected bases; and applying a variant caller model to the cell population features derived from the aggregated sequence reads to identify one or more variants across the cell population. In various embodiments, the single cell features comprise contextual sequences around the base of interest, sequencing depth of the base of interest, allele frequency of the base of interest, and allele frequency of bases in a window around the base of interest.

In various embodiments, identifying a base of interest of the sequence reads comprises: applying a transition matrix comprising likelihoods of transition between reference bases and mismatched bases to a probability of observing a proportion of nucleotide bases across the sequence reads for a mismatched base. In various embodiments, identifying a base of interest of the sequence reads comprises: determining the probability of observing proportion of nucleotide bases across the sequence reads for the mismatched base; and comparing the determined probability to a likelihood of transition from the transition matrix. In various embodiments, responsive to the determined probability being greater than the likelihood of transition, the mismatched base is identified as a base of interest. In various embodiments, the transition matrix is generated using training data comprising sequence reads derived from one or more sample populations of cells. In various embodiments, the transition matrix is generated using the plurality of sequence reads from cells of the cell population. In various embodiments, the likelihoods of transition in the transition matrix are dynamically updated as sequence reads of the one or more cells of the cell population are corrected.

In various embodiments, the error correction model is a neural network. In various embodiments, the error correction model is a deep learning neural network comprising one or more layers that learn motifs and local sequence contexts around a base of interest. In various embodiments, correcting one or more sequence reads of the plurality of sequence reads derived from the cell results comprises correcting at least 25% of bases of interest that differ from a reference base.

In various embodiments, the cell population features comprise one or more of percentage of heterozygous calls, median variant allele frequency (VAF) of heterozygous calls, median genotype quality of heterozygous calls, median read depth of heterozygous calls, percentage of homozygous calls, median VAF of homozygous calls, median genotype quality of homozygous calls, median read depth of homozygous calls, percentage of reference calls, coefficient of variation (CV) of read depth for homozygous calls, CV of read depth for heterozygous calls, CV of genotype quality of homozygous calls, CV of genotype quality of heterozygous calls, CV of VAF for homozygous calls, CV of VAF for heterozygous calls, difference between mean and median VAF for homozygous calls, difference between mean and median VAF for heterozygous calls, and amplicon GC percentage.

In various embodiments, the variant caller model predicts at least one of a heterozygous variant of interest or a homozygous variant of interest. In various embodiments, the variant caller model further predicts indeterminate variants. In various embodiments, the variant caller model is trained using training data comprising sequence reads derived from one or more cell lines and indications of known heterozygous or homozygous variants present in the one or more cell lines.

In various embodiments, the application of the error correction model and the variant caller model achieves at least a two-fold increase in true variant positive predictive value at a limit of detection (LOD) of 0.5% in comparison to a conventional GTAK variant caller. In various embodiments, the application of the error correction model and the variant caller model achieves a true variant positive predictive value of at least 0.6 at a limit of detection (LOD) of 0.5%. In various embodiments, the reference base is determined from a reference genome sequence. In various embodiments, the reference base is determined from one or more sequence reads obtained from a control cell.

### BRIEF DESCRIPTION OF THE SEVERAL VIEWS OF THE DRAWINGS

These and other features, aspects, and advantages of the present invention will become better understood with regard to the following description, and accompanying drawings, where:

Figure (FIG.) 1 depicts an overall system environment including a cell analysis workflow device and a base caller device for identifying variant calls, in accordance with an embodiment.

FIG. 2 is a block diagram of separate modules of a base caller device, in accordance with an embodiment.

FIG. 3A is a flow diagram for correcting sequence reads derived from single cells, in accordance with an embodiment.

FIG. 3B depicts a flow diagram for calling variants of a cell population using corrected sequence reads, in accordance with an embodiment.

FIG. 4A depicts the implementation of the error correction model, in accordance with an embodiment.

FIG. 4B depicts the implementation of the variant caller model, in accordance with an embodiment.

FIG. 5 depicts an example computing device for implementing system and methods described in reference to FIGS. 1-4.

FIG. 6 depicts an example distribution of base errors, where a majority of base errors are observed in only one cell.

FIG. 7 is an example depiction of a transition matrix.

FIGS. 8A and 8B are example depictions of a pileup of six sequence reads across different positions.

FIG. 9A depicts example input and output of the error correction model.

FIG. 9B depicts an example of correcting a base of interest using probabilities predicted by the error correction model.

FIG. 10 demonstrates correction of 20-35% bases across four different cell populations as a result of implementing the error correction model.

FIG. 11 demonstrates improved positive predictive value of true variants following implementation of the error correction model and the variant caller model.

### DETAILED DESCRIPTION

### Definitions

Terms used in the claims and specification are defined as set forth below unless otherwise specified.

The phrases “mismatched base” and “alternate base” are used interchangeably and refers to a base at a position that differs from a known reference base at the same position. In some scenarios, a mismatched base is erroneously identified (e.g., erroneously identified during sequencing). An erroneous identification of a base can arise from various sources such as PCR errors, sequencing errors, sequencing alignment errors, and/or correction errors. To provide an example, a known base at a reference position can be adenine (A). A mismatched base or alternate base refers to base other than adenine (A) at the same position (e.g., the base is any one of guanine (G), cytosine (C), or thymine (T)).

The phrase “reference base” refers to a known base with a known nucleotide base. In one embodiment, the reference base is determined from a reference genome sequence. In one embodiment, the reference base is determined from one or more sequence reads obtained from a control cell.

The phrase “error correction model” refers to a prediction model or a machine-learned model that is implemented to analyze a base of interest such that the base of interest can be corrected. Generally, the error correction model is implemented to analyze a base of interest in a cell-specific manner. In one embodiment, the error correction model analyzes a pileup generated for the base of interest, the pileup quantifying bases of sequence reads that are derived from a single cell. In such embodiments, the sequence reads from the single cell that include the base of interest can be corrected together.

The phrase “base of interest” refers to a base across sequence reads derived from a cell that is mismatched in comparison to a reference base. In various embodiments, a base of interest as likely an erroneous base by applying a transition matrix. Generally, a pileup generated for the base of interest is analyzed by the error correction model to determine whether the base of interest is likely an erroneous base.

The phrase “single cell features” refers to features relevant to a base of interest in sequence reads of a single cell. In various embodiments, the single cell features are analyzed by the error correction model to determine a distribution of probabilities corresponding to the four nucleotide bases (adenine, guanine, cytosine, and thymine), the distribution of probabilities representing likelihoods that the base of interest is one of the four nucleotide bases. Examples of single cell features include contextual sequences around the base of interest, sequencing depth of the base of interest, allele frequency of the base of interest, and allele frequency of bases in a window around the base of interest.

The phrase “variant caller model” refers to a prediction model or a machine-learned model that is implemented to call variants of a cell population. The variant caller model analyzes cell population features derived from corrected sequence reads across a cell population, the sequence reads having undergone error correction (e.g., corrected using the error correction model). In one embodiment, the variant caller model receives the cell population features as input and predicts a classification for a candidate variant. In one embodiment, the variant caller model extracts cell population features from sequence reads that were previously corrected and predicts a classification for a candidate variant based on the extracted cell population features.

The phrase “cell population features” refers to features relevant to a candidate variant derived from corrected sequence reads across a cell population. Cell population features are analyzed by the variant caller model to predict true variants of the cell population. Examples of cell population features include percentage of heterozygous calls, median variant allele frequency (VAF) of heterozygous calls, median genotype quality of heterozygous calls, median read depth of heterozygous calls, percentage of homozygous calls, median VAF of homozygous calls, median genotype quality of homozygous calls, median read depth of homozygous calls, percentage of reference calls, coefficient of variation (CV) of read depth for homozygous calls, CV of read depth for heterozygous calls, CV of genotype quality of homozygous calls, CV of genotype quality of heterozygous calls, CV of VAF for homozygous calls, CV of VAF for heterozygous calls, difference between mean and median VAF for homozygous calls, difference between mean and median VAF for heterozygous calls, and amplicon GC percentage.

The phrase “candidate variant” refers to a base across sequence reads of a cell population that are mismatched in comparison to a reference base. Generally, a variant caller model is implemented to determine whether the candidate variant is a true variant, such as a homozygous variant or a heterozygous variant.

The phrase “true variant” refers to a genetic variant that is present in one or more cells of a cell population.

### Overview

Embodiments described herein refer to an improved variant caller that performs a cell-specific error correction of bases and further performs an identification of variants using the error corrected sequence reads. In various embodiments, the cell-specific error correction involves implementing an error correction model and the identification of variants involves implementing a variant caller model. Altogether, the variant caller method described herein achieves higher accuracy in calling true variants that are present in cells in comparison to conventional variant caller methods (e.g., Genome Analysis Toolkit (GATK)) that employ hard cutoffs as opposed to an error correction model and/or variant caller model. Further description regarding hard filters used in the GATK is found in De Summa, S., Malerba, G., Pinto, R. et al. GATK hard filtering: tunable parameters to improve variant calling for next generation sequencing targeted gene panel data. BMC Bioinformatics 18, 119 (2017), which is incorporated by reference in its entirety.

Reference is made to FIG. 1, which depicts an overall system environment 100 including a cell analysis workflow device 120 and a base caller device 130 for variant calling, in accordance with an embodiment. A cell population 110 is obtained. In various embodiments, the cell population 110 can be isolated from a test sample obtained from a subject or a patient. In various embodiments, the cell population 110 includes healthy cells taken from a healthy subject. In various embodiments, the cell population 110 includes diseased cells taken from a subject. In one embodiment, the cell population 110 includes cancer cells taken from a subject previously diagnosed with cancer. For example, cancer cells can be tumor cells available in the bloodstream of the subject diagnosed with cancer. As another example, cancer cells can be cells obtained through a tumor biopsy.

The cell analysis workflow device 120 refers to a device that processes cells and generates nucleic acids for sequencing. In various embodiments, the cell analysis workflow device 120 refers to a system comprising one or more devices that process cells and generate nucleic acids for sequencing. In various embodiments, the cell analysis workflow device 120 is a workflow device that generates nucleic acids from single cells, thereby enabling the subsequent identification of sequence reads and individual cells from which the sequence reads originated. In various embodiments, the cell analysis workflow device 120 can perform single-cell processing by encapsulating individual cells into emulsions, lysing cells within emulsions, performing cell barcoding of cell lysate in emulsions, and performing a nucleic amplification reaction in emulsions. Thus, amplified nucleic acids can be collected and sequenced. Further description of example embodiments of single-cell workflow processes is described in U.S. application Ser. No. 14/420,646, which is hereby incorporated by reference in its entirety.

In particular embodiments, the cell analysis workflow device 120 can be any of the Tapestri™ Platform, inDrop™ system, Nadia™ instrument, or the Chromium™ instrument. In various embodiments, the cell analysis workflow device 120 includes a sequencer for sequencing the nucleic acids to generate sequence reads.

The base caller device 130 is configured to receive the sequence reads from the cell analysis workflow device 120 and to process the sequence reads to call one or more variants 140. In various embodiments, the base caller device 130 is communicatively coupled to the cell analysis workflow device 120, and therefore, directly receives the sequence reads from the cell analysis workflow device 120. The base caller device 130 error corrects bases of interest in the sequence reads and then calls likely variants in the cell population 110. In particular embodiments, the base caller device 130 corrects bases of interest in the sequence reads through a cell-specific workflow process and subsequently calls variants across the cell population using the corrected sequence reads. Altogether, this two-step process of cell-specific error correction and cell population variant calling enables more accurate variant calls 140 across the cell population 110.

### Base Caller Device

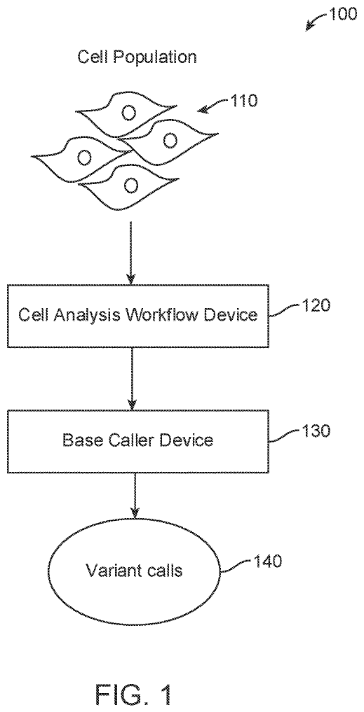

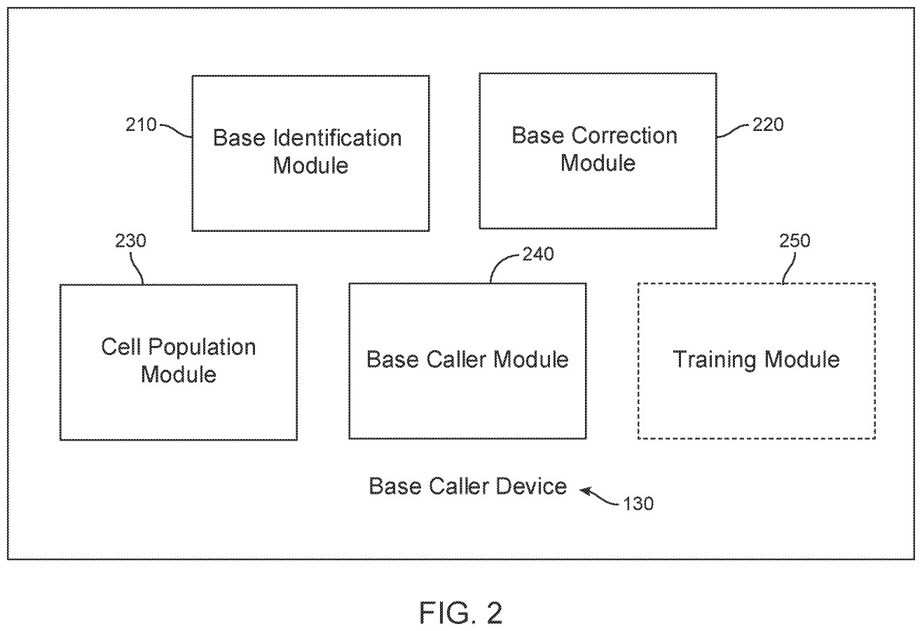

FIG. 2 is a block diagram of a base caller device 130, in accordance with the embodiment described in FIG. 1. As shown in FIG. 2, the base caller device 130 includes a base identification module 210, a base correction module 220, a cell population module 230, a base caller module 240, and a training module 250. In some embodiments, the modules of the base caller device 130 can be arranged differently than the embodiment shown in FIG. 2. For example, the training module 250 (as shown in dotted lines) can be implemented by a device other than the base caller device 130 and the methods described below in regards to the training module 250 can be performed by the other device.

Generally, the base identification module 210 analyzes sequence reads derived from individual cells and identifies one or more bases of interest that are mismatched in comparison to reference bases. The base identification module 210 identifies bases of interest on a per-cell basis. For example, the base identification module 210 analyzes sequences reads from a first cell to determine bases of interest of sequence reads from the first cell. The base identification module 210 further analyzes sequence reads from a second cell to determine bases of interest of sequences reads from the second cell and so on. Sequence reads from different cells can be differentiated from one another using barcode technologies, examples of which are further described in PCT/US2016/016444, which is hereby incorporated by reference in its entirety. Furthermore, for each cell, the base identification module 210 generates a pileup of sequence reads corresponding to the bases of interest of the cell and provides the pileups to the base correction module 220 for determining whether to correct any of the bases of interest.

In various embodiments, the base identification module 210 obtains sequence reads aligned to a reference genome. As an example, the base identification module 210 can obtain sequence reads in a readable file format, such as a SAM (sequence alignment map) file format or BAM (binary alignment map) file format.

Given the aligned sequence reads, the base identification module 210 identifies one or more bases of interest across the sequence reads derived from the cell. In various embodiments, the base identification module 210 analyzes each mismatched base to determine whether a mismatched base is a base of interest.

In various embodiments, to identify a base of interest, the base identification module 210 applies a filter in determining whether at least a threshold number of sequence reads at the position from the cell have a particular nucleotide base that differs from the reference base at the position. In various embodiments, if more than a threshold number of sequence reads at the position have a nucleotide base that differs from the reference base, the base identification module 210 identifies the base as a base of interest for subsequent correction.

In various embodiments, the threshold number of sequence reads at the position is a fixed value. In various embodiments, the threshold number of sequence reads is greater than 1000, greater than 2000, greater than 3000, greater than 4000, greater than 5000, greater than 6000, greater than 7000, greater than 8000, greater than 9000, greater than 10,000, greater than 20,000, greater than 30,000, greater than 40,000, greater than 50,000, greater than 75,000, greater than 100,000, greater than 150,000, greater than 200,000, greater than 250,000, or greater than 500,000 sequence reads. In various embodiments, the threshold number of sequence reads is greater than 5% of total sequence reads at the position from the cell, greater than 10% of total sequence reads at the position from the cell, greater than 20% of total sequence reads at the position from the cell, greater than 30% of total sequence reads at the position from the cell, greater than 40% of total sequence reads at the position from the cell, greater than 50% of total sequence reads at the position from the cell, greater than 60% of total sequence reads at the position from the cell, greater than 70% of total sequence reads at the position from the cell, greater than 75% of total sequence reads at the position from the cell, greater than 80% of total sequence reads at the position from the cell, greater than 85% of total sequence reads at the position from the cell, greater than 90% of total sequence reads at the position from the cell, or greater than 95% of total sequence reads at the position from the cell.

In various embodiments, the base identification module 210 identifies a base of interest by applying a transition matrix. In such embodiments, applying a transition matrix includes comparing a probability of the transition matrix to a probability reflecting the likelihood of observing a proportion of nucleotide bases of the sequence reads.

Referring first to the transition matrix includes probabilities representing frequencies of transition between nucleotides of reference bases and observed nucleotides of bases at a particular position. Generally, the probabilities representing frequencies of transitions in the transition matrix enable the base identification module 210 to distinguish between a mismatched base that is likely due to an error (PCR error, sequencing error, etc.) and a mismatched base that did not arise due to error.

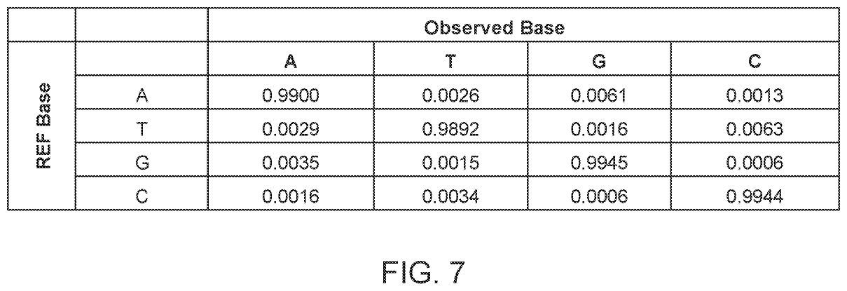

In various embodiment, the transition matrix includes, for a given reference base (e.g., A, C, G, or T), probabilities that the reference base is observed as a different base in the sequence read. In various embodiments, the transition matrix includes 12 probability values (e.g., 3 probability values reflecting transition from reference bases to mismatched bases). In various embodiments, the transition matrix includes 16 probability values. This includes a probability for each reference base that the observed base in sequence reads matches the reference base. An example transition matrix is described below in FIG. 7.

FIG. 7 is an example depiction of a transition matrix. Here, the transition matrix includes designations of the reference base (e.g., “REF” on the y-axis) and the observed base (e.g., “Observed Base” on the x-axis). Each cell in the transition matrix includes a likelihood value representing the probability that a nucleotide for a reference base is observed as a nucleotide for an observed base. For example, the first row of the transition matrix indicates that for a known adenine reference base “A,” the probability of the observed base matching the reference adenine base is 99% (first row). However, in some scenarios, a reference adenine base is differently observed in a sequence read. For example, for a known adenine reference base “A,” the probability of an observed base mismatching the reference adenine base is 0.26% (first row, second column indicating an observed thymine base), 0.61% (first row, third column indicating an observed guanine base), and 0.13% (first row, fourth column indicating an observed cytosine base).

In some embodiments, the transition matrix is previously generated from one or more prior samples. A prior sample can include cells of a cell population or can include cells of a mixture of cell populations. In such embodiments, the transition matrix serves as a reference that can be applied across different samples. Thus, the transition matrix can be used to identify bases of interest for different samples. In various embodiments, the base identification module 210 generates a transition matrix for each sample that undergoes the variant calling process. Therefore, in such embodiments, the base identification module 210 applies a different transition matrix for each sample when identifying bases of interest. This may be preferable in some scenarios as errors can arise in a sample-dependent manner.

In various embodiments, the base identification module 210 generates a transition matrix using at least in part the same sequence reads that the base identification module 210 is analyzing to identify a base of interest. In such embodiments, as bases of interest are corrected (e.g., corrected using the error correction model as described below), the base identification module 210 can dynamically update the probabilities in the transition matrix to reflect the new nucleotide base of the corrected base. As an example of how the base identification module 210 generates a transition matrix, for a position with a reference base of “A,” the base identification module 210 determines a proportion of sequence reads that have any of the four nucleotide bases (A, C, T, or G) at the position. Therefore, the base identification module 210 quantifies a probability distribution across the four nucleotide bases for the position with the reference base of “A.” The base identification module 210 can determine probabilities of transition for reference nucleotide bases of “C,” “T,” and “G.”

In various embodiments, the base identification module 210 determines the probability reflecting the likelihood of observing the proportion of nucleotide bases for the position across the sequence reads. In some embodiments, the probability can be denoted as:

P(Adenine=W,Cytosine=X,Guanine=Y,Thymine=Z|N reads)

where W is the number of observed sequence reads with adenine nucleotide bases at the position,
where X is the number of observed sequence reads with cytosine nucleotide bases at the position,
where Y is the number of observed sequence reads with thymine nucleotide bases at the position,
where Z is the number of observed sequence reads with thymine nucleotide bases at the position, and where N is the total number of observed sequence reads at the position.

In some embodiments, the probability reflects the likelihood of observing the proportion of mismatched nucleotide bases for the position across the sequence reads. Here, the probability can be denoted as:

P(Base 1=X,Base 2=Y,Base 3=Z|N reads)

where base 1, base 2, and base 3 refer to nucleotide bases that do not match with the reference base, where X is the number of observed sequence reads with base 1 at the position, where Y is the number of observed sequence reads with base 2 at the position, where Z is the number of observed sequence reads with base 3 at the position, and where N is the total number of observed sequence reads at the position.

The base identification module 210 compares the probability reflecting the likelihood of observing the proportion of nucleotide bases for the position to a probability of the transition matrix. In various embodiments, if comparison yields that the probability reflecting the likelihood of observing the proportion of nucleotide bases is greater than the probability of the transition matrix, the base identification module 210 identifies the base as a base of interest. Thus, the base of interest can subsequently undergo correction. If comparison yields that the probability reflecting the likelihood of observing the proportion of nucleotide bases is less than the probability of the transition matrix, the base identification module 210 does not identify the base as a base of interest. Thus, the base does not undergo correction and remains a mismatched base.

As an overall example of identifying a base of interest, the base identification module 210 may identify that a majority of sequence reads at a position have a mismatch of adenine (reference base) to guanine (observed base). The transition matrix includes a probability reflecting a likelihood of a transition from a reference adenine base to an observed guanine base. Assume this probability is 0.01. The base identification module 210 may determine the probability of observing the proportion of nucleotide bases other than the reference base (e.g., observing guanine, cytosine, or thymine nucleotide bases) is 0.05. The base identification module 210 compares the probability of observing the proportion of nucleotide bases (0.05) to the probability of the transition matrix (0.01). Here, given that the probability of observing the proportion of nucleotide bases (0.05) is greater than the probability of the transition matrix (0.01), the base identification module 210 identifies the base as a base of interest.

In various embodiments, having identified bases of interest, the base identification module 210 generates a pileup of sequence reads for each base of interest. Specifically, the base identification module 210 generates a pileup including sequence reads that include bases that are located X positions upstream and Y positions downstream of the base of interest. In various embodiments, X and Y are the same value. In other embodiments, X and Y are different values. In various embodiments, X can be 1 position, 2 positions, 3 positions, 4 positions, 5 positions, 6 positions, 7 positions, 8 positions, 9 positions, 10 positions, 15 positions, 20 positions, 25 positions, 30 positions, 40 positions, 50 positions, 60 positions, 70 positions, 80 positions, 90 positions, 100 positions, 110 positions, 120 positions, 130 positions, 140 positions, or 150 positions upstream of the base of interest. In various embodiments, Y can be 1 position, 2 positions, 3 positions, 4 positions, 5 positions, 6 positions, 7 positions, 8 positions, 9 positions, 10 positions, 15 positions, 20 positions, 25 positions, 30 positions, 40 positions, 50 positions, 60 positions, 70 positions, 80 positions, 90 positions, 100 positions, 110 positions, 120 positions, 130 positions, 140 positions, or 150 positions downstream of the base of interest.

In various embodiments, the base identification module 210 generates a pileup such that for the positions located upstream and downstream to the position of the base of interest, the pileup includes probabilities indicating the proportion of sequence reads that had one of the four nucleotide bases (e.g., adenine, guanine, cytosine, or thymine). For example, the pileup may be embodied as a matrix such that for each position in the pileup, the matrix includes probabilities that identify the proportion of sequence reads that had a corresponding adenine, guanine, cytosine, or thymine at the position.

The base correction module 220 applies an error correction model to determine the likely nucleotide of the base of interest. Therefore, the base correction module 220 can correct the base of interest across one or more sequence reads derived from the cell. The corrected sequence reads represent improved sequence reads that can subsequently be used to call true variants. Generally, The base correction model 220 corrects sequence reads through a cell-specific process. Here, the base correction model 220 may correct a base of interest in sequence reads for a first cell, but may not correct the same base in sequence reads for a second cell. As errors (e.g., PCR errors, sequencing errors, sequencing alignment errors, or correction errors) can arise in individual cells, the methods performed by the base correction model 220 enable the correction of sequence reads on a per-cell basis to address these errors.

The base correction module 220 receives pileups generated for bases of interest. In one embodiment, the base correction module 220 applies a pileup for a base of interest as input to an error correction model. Here, the error correction model can extract and analyze single cell features of the pileup including contextual sequences around the base of interest, sequencing depth of the base of interest, allele frequency of the base of interest, and allele frequency of bases in a window around the base of interest. In various embodiments, a “window” refers to X bases located upstream to the base of interest and Y bases located downstream to the base of interest. In various embodiments, X and Y can, independent of each other, be 2 bases, 3 bases, 4 bases, 5 bases, 6 bases, 7 bases, 8 bases, 9 bases, 10 bases, 20 bases, 30 bases, 40 bases, 50 bases, 60 bases, 70 bases, 75 bases, 80 bases, 90 bases, 100 bases, 150 bases, 200 bases, 300 bases, 400 bases, or 500 bases. As an example, the error correction model can be a neural network (e.g., deep learning neural network) that extracts single cell features from the pileup and analyzes the single cell features. In some embodiments, the base correction module 220 performs a feature extraction process to extract the single cell features from the pileup. In such embodiments the single cell features can be provided as input to the error correction model. In various embodiments, the error correction model outputs a distribution of probabilities corresponding to the four nucleotide bases (adenine, guanine, cytosine, and thymine), the distribution of probabilities representing likelihoods that the base of interest is one of the four nucleotide bases based on the analyzed single cell features.

In various embodiments, the base correction model 220 corrects a base of interest to a different nucleotide base based on the distribution of probabilities outputted by the error correction model. In one embodiment, the base correction model 220 corrects a base of interest to a nucleotide base that has the highest probability amongst the probabilities in the distribution outputted by the error correction model. Here, the corrected nucleotide base represents the likely base that is present in the cell. To correct a base of interest to a different nucleotide base, the base correction model 220 corrects one or more sequence reads including the base of interest to reflect the correct nucleotide base. Altogether, the base correction model 220 regenerates corrected sequence reads with corrected nucleotide bases that more accurately reflect sequences of the cell.

In various embodiments, the base correction model 220 corrects all sequence reads derived from a single cell with the base of interest, such that after the correction, the corrected sequence reads include the correct base. In various embodiments, the base correction model 220 corrects a portion of sequence reads derived from a single cell with the base of interest. For example, some of the sequence reads with the base of interest may have the correct base and therefore, need not be corrected. As another example, some sequence reads with the base of interest may be low confidence reads and can be discarded as opposed to corrected. In various embodiments, the base correction model 220 generates corrected sequence reads in a readable file format, such as a BAM file format or a SAM file format.

The cell population module 230 determines cell population features from the corrected sequence reads across the cell population. Generally, the cell population module 230 analyzes the corrected sequence reads, which were organized on a per-cell basis, and determines cell population features that are descriptive of the cell population.

The cell population module 230 identifies one or more candidate variants across the cell population that remain after the sequence reads of cells have been error corrected. In various embodiments, candidate variants include all variants that remain after the sequence reads have been corrected. In various embodiments, the cell population module 230 performs a filter such that candidate variants are a subset of all variants that remain after the sequence reads have been corrected. For example, the cell population module 230 identifies a candidate variant at a particular position if the base satisfies one or more criteria. In various embodiments, the one or more criteria serve as a hard cutoff that include one or both of 1) a minimum allele frequency and 2) a minimum number of cells having a mismatched base at the position.

In various embodiments, to determine cell population features across the cell population, the cell population module 230 aggregates corrected sequence reads on a per cell basis, and then determines cell population features across the cell population using the aggregated sequence reads. For example, for each cell, the cell population module 230 can quantify the proportion of sequence reads with particular nucleotide bases (e.g., A, C, T, or G) at each position. The cell population module 230 then determines cell population features across the cell population by analyzing the quantified proportion of sequence reads.

In various embodiments, the cell population module 230 determines cell population features for each of one or more candidate variants. As a specific example, a cell population feature may be a percentage of heterozygous calls for a particular candidate variant (e.g., percentage of cells where, at a particular position, a first copy of the candidate variant is mismatched in comparison to a reference base and the second copy of the candidate variant matches the reference base). Thus, for a cell, the cell population module 230 aggregates corrected sequence reads for the cell and determines whether the candidate variant for the cell is a heterozygous call. The cell population module 230 repeats this process across cells of the cell population to derive the percentage of cells with a heterozygous call corresponding to the candidate variant. For additional candidate variants, the cell population module 230 determines the percentage of cells that have heterozygous copies of each of the additional candidate variants.

Examples of cell population features include, but are not limited to, percentage of heterozygous calls, median variant allele frequency (VAF) of heterozygous calls, median genotype quality of heterozygous calls, median read depth of heterozygous calls, percentage of homozygous calls, median VAF of homozygous calls, median genotype quality of homozygous calls, median read depth of homozygous calls, percentage of reference calls, coefficient of variation (CV) of read depth for homozygous calls, CV of read depth for heterozygous calls, CV of genotype quality of homozygous calls, CV of genotype quality of heterozygous calls, CV of VAF for homozygous calls, CV of VAF for heterozygous calls, difference between mean and median VAF for homozygous calls, difference between mean and median VAF for heterozygous calls, and amplicon GC percentage.

The base caller module 240 applies a variant caller model to predict one or more true variants of a cell population. In various embodiments, the base caller module 240 provides, as input, cell population features for a candidate variant to the variant caller model. The variant caller model analyzes the cell populations feature and outputs a prediction for the candidate variant.

In various embodiments, the variant caller is a classifier that outputs a classification for the candidate variant out of multiple possible classifications. In some embodiments, the variant caller model is a classifier that outputs one of two classifications for the candidate variant. As an example, the variant caller model can output a classification of a true variant or a false positive variant. As another example, the variant caller model can output a classification as to a type of a true variant, such as one of a homozygous variant or a heterozygous variant. In some embodiments, the variant caller model is a classifier that outputs one of more than two possible classifications for the candidate variant. As an example, the variant caller model can output a classification of a homozygous variant, a heterozygous variant, or a false positive variant. In some embodiments, the variant caller model outputs a classification of an indeterminate variant. An indeterminate variant can represent a low confidence call that may require additional analysis to confirm as to whether the indeterminate variant is a true variant. In some embodiments, the variant caller model outputs a classification of a non-variant (e.g., a false positive variant).

The training module 250 generally implements methods for generating one or both of the error correction model and the variant caller model. In various embodiments, the training module 250 is implemented by a device or system other than the base caller device 130. For example, the training module 250 can be implemented by a third party. In such a scenario, the third party generates one or both of the error correction model and the variant caller model. The third party can then provide one or both of the trained error correction model and the trained variant caller model to the base caller device 130.

In various embodiments, the training module 250 trains the error correction model. The training module 250 can employ a machine learning implemented method to train the error correction model, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the training module 250 employs supervised learning algorithms, unsupervised learning algorithms, semi-supervised learning algorithms (e.g., partial supervision), transfer learning, multi-task learning, or any combination thereof to train the error correction model.

The training module 250 trains the error correction model using error correction training samples. In various embodiments, the error correction training samples include training sequence reads derived from individual cells. Such training samples can be expressed in a commonly used file format such as a SAM or BAM file format. In various embodiments, training sequence reads in the error correction training sample includes sequence reads with a known base of interest that is mismatched in comparison to a reference base. These training sequence reads can be derived from individual cells that are known to have a genetic variant at the position of the known base of interest.

In various embodiments, the error correction training samples can be labeled with reference ground truths indicating a known base of the genetic variant present in a cell. In various embodiments, a label for a known base can be an integer (e.g., 0, 1, 2, and 3) where each integer value is indicative of a nucleotide base (e.g., one of A, C, T, or G) for the known base. In various embodiments, a label for a known base can be structured as a vector (e.g., a 1×4 matrix such as [0, 0, 0, 1]). In such an example, each cell in the matrix corresponds to one of the four nucleotide bases. A value of “0” indicates that the corresponding nucleotide base is not the known base, whereas a value of “1” indicates that the corresponding nucleotide base is the known base.

In various embodiments, an error correction training sample includes: 1) one or more training sequence reads derived from a cell with a base of interest and 2) a label indicating the known base. In various embodiments, the training module 250 creates training pileups of varying sizes using the one or more training sequence reads of the error correction training sample. Thus, the error correction model can be iteratively trained using pileups derived from training sequence reads of a training sample. The parameters of the error correction model are adjusted during training iterations such that the error correction model can better predict a distribution of probabilities for a base of interest.

In various embodiments, the training module 250 trains the variant caller model. The training module 250 can employ a machine learning implemented method to train the variant caller model, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the training module 250 employs supervised learning algorithms, unsupervised learning algorithms, semi-supervised learning algorithms (e.g., partial supervision), transfer learning, multi-task learning, or any combination thereof to train the variant caller model.

The training module 250 trains the variant caller model using variant caller training samples. In various embodiments, the variant caller training samples include training sequence reads including known variants or known reference bases. In various embodiments, the variant caller training samples include cell population features derived from training sequence reads.

The variant caller training samples can be labeled with reference ground truths indicating a classification of variants. In one embodiment, the reference ground truths differentiate between a true variant and a false positive variant. In one embodiment, the reference ground truths differentiate between different true variants such as a homozygous variant and a heterogeneous variant. In one embodiment, the reference ground truths differentiate between a homozygous variant, a heterozygous variant, and reference base (e.g., non-variant).

In various embodiments, the labels of the variant caller training samples can be previously determined and/or confirmed through other sequencing methods, such as bulk sequencing methods. In various embodiments, labels of the variant caller training samples can be previously determined at least in part based on known genetic variants that are present in certain cell lines. In various embodiments, a label can be a binary value (e.g., a 0 or 1 value) that is indicative of whether the variant is true variant or a false positive variant. In some embodiments, a label can be different integer values (e.g., 0, 1, 2, 3, etc.) depending on the number of classifications that the variant caller model is designed to predict. For example, for a variant caller model that predicts homozygous variants, heterozygous variants, and reference bases (e.g., non-variants), labels can be three integer values (e.g., 0, 1, and 2), each integer value corresponding on one of the classifications.

In various embodiments, each variant caller training sample includes: 1) training sequence reads of a cell population a with known reference base or known variant and 2) a label indicating the presence of the known reference base or known variant corresponding to the training sequence read. Thus, the variant caller model can be iteratively trained using each variant caller training sample. In various embodiments, the parameters of the variant caller model are adjusted during training iterations such that the variant caller model can better predict whether sequence reads of a cell population have a reference base or a true variant.

### Methods for Calling Variants of a Cell Population

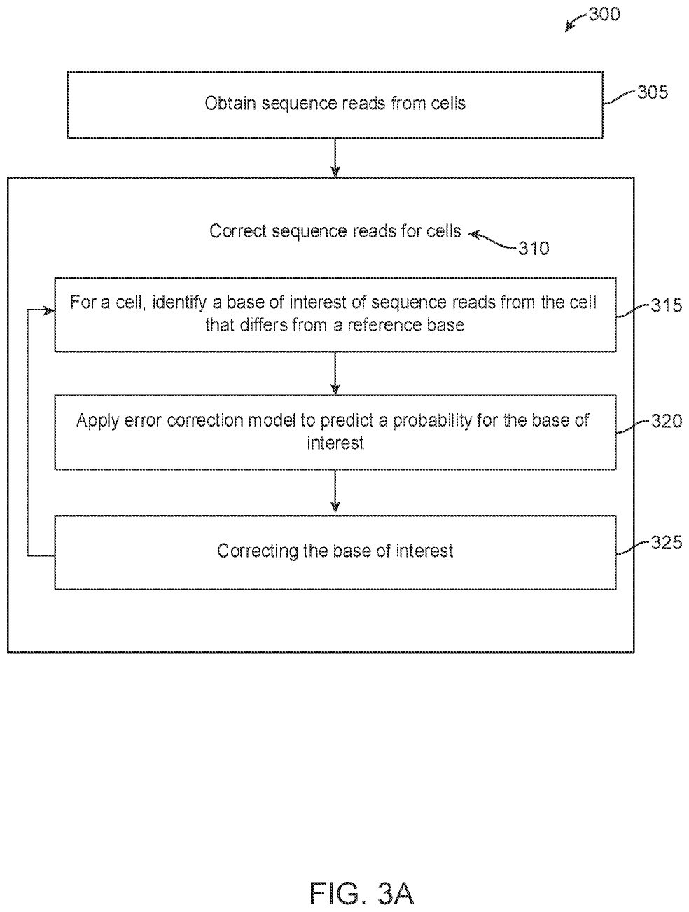

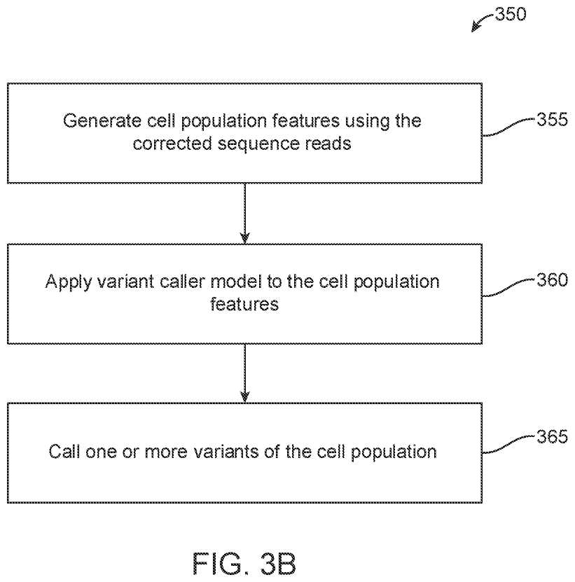

Reference is now made to flow diagrams 300 and 350 shown in FIGS. 3A and 3B, which describe the two step process involving 1) error correction of bases in sequence reads through a cell-specific process and 2) variant calling across cell populations using error corrected sequence reads.

FIG. 3A is a flow diagram 300 for correcting sequence reads derived from single cells, in accordance with an embodiment. At step 305, sequence reads are obtained from cells. In various embodiments, sequence reads from one cell are distinguishable from sequence reads from another cell (e.g., previously distinguished using barcode technologies). Additionally, such sequence reads can be aligned to a reference genome.

At step 310, sequence reads for cells are corrected by correcting erroneous bases in the sequence reads. Step 310 is a cell-specific process that involves steps 315, 320, and 325. In various embodiments, steps 315, 320, and 325 are performed in parallel for each of one or more cells of a cell population. In various embodiments, steps 315, 320, and 325 are sequentially performed for each of one or more cells of a cell population. Altogether, the steps 315, 320, and 325 result in the generation of corrected sequence reads for each of one or more cells of the cell population.

Step 315 involves identifying a base of interest of sequence reads from a cell, the base of interest differing from a reference base. In various embodiments, identifying a base of interest involves applying a transition matrix to determine whether the base mismatch is likely due to an error. Applying a transition matrix involves comparing a probability of the transition matrix to a probability reflecting the likelihood of observing a proportion of nucleotide bases of the sequence reads.

Step 320 involves applying an error correction model to predict a probability for the base of interest. In various embodiments, the error correction model analyzes single cell features derived from a pileup generated for the base of interest and outputs a distribution of probabilities.

Step 325 involves correcting the base of interest. Here, the base of interest is corrected to a different base that corresponds to the predicted probability. One or more sequence reads from the cell that contains the base of interest can be corrected to the different base.

FIG. 3B depicts a flow diagram 350 for calling variants of a cell population using corrected sequence reads, in accordance with an embodiment. Here, the steps of 355, 360, and 365 are conducted at the cell population level, thereby enabling the calling of true variants across the cell population.

Step 355 involves generating cell population features from the corrected sequence reads across the cell population. In various embodiments, step 355 involves generating cell population features for candidate variants in the cell population using the corrected sequence reads. Step 360 involves applying the variant caller model to the cell population features. In various embodiments, the cell population features for a candidate variant are applied as input to the variant caller model. The variant caller model can be repeatedly applied for different candidate variants to determine whether each candidate variant is a likely true variant.

At step 365, based on the output of the variant caller model, one or more variants across the cell population are called. In various embodiments, calling a variant comprises calling a candidate variant as one of a homozygous variant, a heterozygous variant, or an indeterminate variant.

Altogether, the called variants of the cell population identified through the flow diagrams 300 and 350 represent an improvement beyond conventionally called variants using conventional variant caller pipelines. As such, the called variants can be informative for a variety of applications, examples of which include the characterization of aberrant cells and/or diseases (e.g., cancer).

### Embodiments of Error Correction Model and Variant Correction Model

In particular embodiments, the error correction model and the variant correction model are machine-learned models. Each of the error correction model and the variant correction model may be trained using training data. Following training, the error correction model and the variant correction model can be deployed (e.g., deployed in accordance with the processes described above in reference to FIGS. 3A and 3B).

In various embodiments, one or both of the error correction model the variant correction model is any one of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LS™), bi-directional recurrent networks, deep bi-directional recurrent networks).

In various embodiments, one or both of the error correction model and the variant correction model has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters of one or both of the error correction model and the variant correction model are generally adjusted during training. Examples of model parameters include weights associated with nodes in layers of neural network, support vectors in a support vector machine, and coefficients in a regression model. The model parameters of the machine learning model are trained (e.g., adjusted) using the training data to improve the predictive power of the machine learning model.

In some embodiments, one or both of the error correction model and the variant correction model are parametric models in which one or more parameters of the models define the dependence between the independent variables and dependent variables. In various embodiments, various parameters of parametric-type models are trained to minimize a loss function, the training being conducted through gradient-based numerical optimization algorithms, such as batch gradient algorithms, stochastic gradient algorithms, and the like. In some embodiments, one or both of the error correction model and the variant correction model are non-parametric models in which the model structure is determined from training data and is not strictly based on a fixed set of parameters.

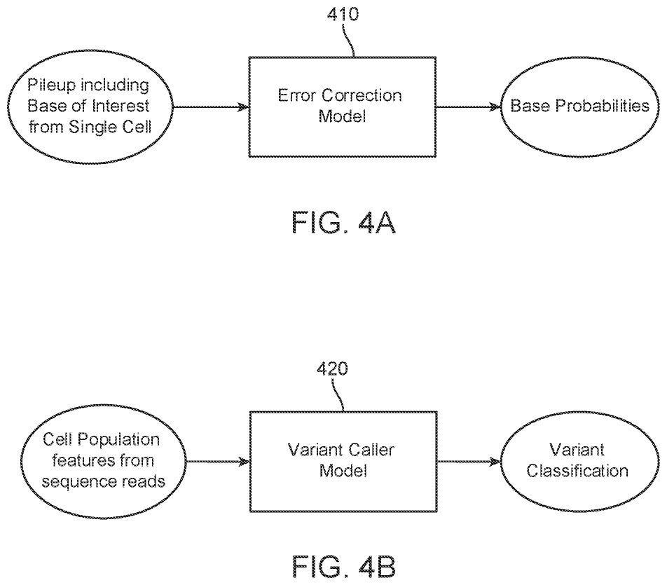

FIG. 4A depicts the implementation of the error correction model 410, in accordance with an embodiment. In this embodiment, the error correction model 410 analyzes a pileup including a base of interest, where the pileup is generated from sequence reads derived from a single cell. In various embodiments, the error correction model 410 analyzes single cell features derived from the pileup generated for the base of interest. Single cell features are features relevant to the base of interest including contextual sequences around the base of interest, sequencing depth of the base of interest, allele frequency of the base of interest, and allele frequency of bases in a window around the base of interest. Based on the single cell features, the error correction model 410 outputs a distribution of base probabilities (e.g., probabilities for one, two, three, or four of adenine, thymine, guanine, and cytosine) that represent likelihoods that the base of interest is another base.

In particular embodiments, the error correction model 410 is a neural network. In some embodiments, the error correction model 410 is a deep learning neural network. The error correction model 410 may be structured with two, three, four, five, six, seven, eight, nine, or ten layers. Layers of the error correction model 410 are comprised of one or more nodes. A node in a layer can be connected to other nodes of other layers, the connection between nodes being associated with parameters. A value at one node may be represented as a combination of the values of nodes connected to the particular node weighted by associated parameters mapped by an activation function associated with the particular node.

FIG. 4B depicts the implementation of the variant caller model, in accordance with an embodiment. In the embodiment shown in FIG. 4B, the variant caller model 420 analyzes cell population features derived from corrected sequence reads across a cell population. The variant caller model 420 outputs a classification for a variant. In some embodiments, the classification for the variant is one of a true variant or a false positive variant. In some embodiments, the classification for the variant is one of a homozygous variant or a heterozygous variant. In some embodiments, the classification for the variant is one of a homozygous variant, a heterozygous variant, or an indeterminate variant.

In some embodiments, the variant caller model 420 receives as input the sequence reads or a pileup of the sequence reads as opposed to the cell population features. In such embodiments, the cell population features need not be extracted from the aggregated reads prior to the implementation of the variant caller model 420. In some embodiments, the aggregated reads can be compiled (e.g., compiled in a pileup) and the pileup of the aggregated reads can be provided as input to the variant caller model 420 to predict a variant classification. For example, a pileup of aggregated reads can be compiled for a base that, after error correction, is a mismatch in comparison to a reference base. The variant caller model 420 analyzes the pileup generated for the base and predicts a variant classification for the base.

In particular embodiments, the variant caller model 420 is a neural network. In some embodiments, the variant caller model 420 is a deep learning neural network. The variant caller model 420 may be structured with two, three, four, five, six, seven, eight, nine, or ten layers. Layers of the variant caller model 420 are comprised of one or more nodes. A node in a layer can be connected to other nodes of other layers, the connection between nodes being associated with parameters. A value at one node may be represented as a combination of the values of nodes connected to the particular node weighted by associated parameters mapped by an activation function associated with the particular node.

### Methods for Sequencing and Read Alignment

Embodiments of the invention disclosed herein involve the sequencing of nucleic acids and the alignment of the sequence reads to a reference genome. In various embodiments, the steps of sequencing nucleic acids and aligning sequence reads to a reference genome is performed by a sequencer, such as a sequencer of the cell analysis workflow device 120, as described above in reference to FIG. 1. Therefore, the sequenced and aligned sequence reads can be analyzed by the base caller device 130 and more specifically, can be analyzed by the base identification module 210 (see FIG. 2) to identify bases of interest.

Sequence reads can be achieved with commercially available next generation sequencing (NGS) platforms, including platforms that perform any of sequencing by synthesis, sequencing by ligation, pyrosequencing, using reversible terminator chemistry, using phospholinked fluorescent nucleotides, or real-time sequencing. As an example, amplified nucleic acids may be sequenced on an Illumina MiSeq platform.

When pyrosequencing, libraries of NGS fragments are cloned, in-situ amplified by capture of one matrix molecule using granules coated with oligonucleotides complementary to adapters. Each granule containing a matrix of the same type is placed in a microbubble of the “water in oil” type and the matrix is cloned amplified using a method called emulsion PCR. After amplification, the emulsion is destroyed and the granules are stacked in separate wells of a titration picoplate acting as a flow cell during sequencing reactions. The ordered multiple administration of each of the four dNTP reagents into the flow cell occurs in the presence of sequencing enzymes and a luminescent reporter, such as luciferase. In the case where a suitable dNTP is added to the 3′ end of the sequencing primer, the resulting ATP produces a flash of luminescence within the well, which is recorded using a CCD camera. It is possible to achieve a read length of more than or equal to 400 bases, and it is possible to obtain 10⁶ readings of the sequence, resulting in up to 500 million base pairs (megabytes) of the sequence. Additional details for pyrosequencing are described in Voelkerding et al., Clinical Chem., 55: 641-658, 2009; MacLean et al., Nature Rev. Microbiol., 7: 287-296; U.S. Pat. Nos. 6,210,891; 6,258,568; each of which is hereby incorporated by reference in its entirety.

On the Solexa/Illumina platform, sequencing data is produced in the form of short readings. In this method, fragments of a library of NGS fragments are captured on the surface of a flow cell that is coated with oligonucleotide anchor molecules. An anchor molecule is used as a PCR primer, but due to the length of the matrix and its proximity to other nearby anchor oligonucleotides, elongation by PCR leads to the formation of a “vault” of the molecule with its hybridization with the neighboring anchor oligonucleotide and the formation of a bridging structure on the surface of the flow cell. These DNA loops are denatured and cleaved. Straight chains are then sequenced using reversibly stained terminators. The nucleotides included in the sequence are determined by detecting fluorescence after inclusion, where each fluorescent and blocking agent is removed prior to the next dNTP addition cycle. Additional details for sequencing using the Illumina platform are found in Voelkerding et al., Clinical Chem., 55: 641-658, 2009; MacLean et al., Nature Rev. Microbiol., 7: 287-296; U.S. Pat. Nos. 6,833,246; 7,115,400; 6,969,488; each of which is hereby incorporated by reference in its entirety.

Sequencing of nucleic acid molecules using SOLiD technology includes clonal amplification of the library of NGS fragments using emulsion PCR. After that, the granules containing the matrix are immobilized on the derivatized surface of the glass flow cell and annealed with a primer complementary to the adapter oligonucleotide. However, instead of using the indicated primer for 3′extension, it is used to obtain a 5′ phosphate group for ligation for test probes containing two probe-specific bases followed by 6 degenerate bases and one of four fluorescent labels. In the SOLiD system, test probes have 16 possible combinations of two bases at the 3′end of each probe and one of four fluorescent dyes at the 5′ end. The color of the fluorescent dye and, thus, the identity of each probe, corresponds to a certain color space coding scheme. After many cycles of alignment of the probe, ligation of the probe and detection of a fluorescent signal, denaturation followed by a second sequencing cycle using a primer that is shifted by one base compared to the original primer. In this way, the sequence of the matrix can be reconstructed by calculation; matrix bases are checked twice, which leads to increased accuracy. Additional details for sequencing using SOLiD technology are found in Voelkerding et al., Clinical Chem., 55: 641-658, 2009; MacLean et al., Nature Rev. Microbiol., 7: 287-296; U.S. Pat. Nos. 5,912,148; 6,130,073; each of which is incorporated by reference in its entirety.

In particular embodiments, HeliScope from Helicos BioSciences is used. Sequencing is achieved by the addition of polymerase and serial additions of fluorescently-labeled dNTP reagents. Switching on leads to the appearance of a fluorescent signal corresponding to dNTP, and the specified signal is captured by the CCD camera before each dNTP addition cycle. The reading length of the sequence varies from 25-50 nucleotides with a total yield exceeding 1 billion nucleotide pairs per analytical work cycle. Additional details for performing sequencing using HeliScope are found in Voelkerding et al., Clinical Chem., 55: 641-658, 2009; MacLean et al., Nature Rev. Microbiol., 7: 287-296; U.S. Pat. Nos. 7,169,560; 7,282,337; 7,482,120; 7,501,245; 6,818,395; 6,911,345; 7,501,245; each of which is incorporated by reference in its entirety.

In some embodiments, a Roche sequencing system 454 is used. Sequencing 454 involves two steps. In the first step, DNA is cut into fragments of approximately 300-800 base pairs, and these fragments have blunt ends. Oligonucleotide adapters are then ligated to the ends of the fragments. The adapter serve as primers for amplification and sequencing of fragments. Fragments can be attached to DNA-capture beads, for example, streptavidin-coated beads, using, for example, an adapter that contains a 5′-biotin tag. Fragments attached to the granules are amplified by PCR within the droplets of an oil-water emulsion. The result is multiple copies of cloned amplified DNA fragments on each bead. At the second stage, the granules are captured in wells (several picoliters in volume). Pyrosequencing is carried out on each DNA fragment in parallel. Adding one or more nucleotides leads to the generation of a light signal, which is recorded on the CCD camera of the sequencing instrument. The signal intensity is proportional to the number of nucleotides included. Pyrosequencing uses pyrophosphate (PPi), which is released upon the addition of a nucleotide. PPi is converted to ATP using ATP sulfurylase in the presence of adenosine 5′ phosphosulfate. Luciferase uses ATP to convert luciferin to oxyluciferin, and as a result of this reaction, light is generated that is detected and analyzed. Additional details for performing sequencing 454 are found in Margulies et al. (2005) Nature 437: 376-380, which is hereby incorporated by reference in its entirety.

Ion Torrent technology is a DNA sequencing method based on the detection of hydrogen ions that are released during DNA polymerization. The microwell contains a fragment of a library of NGS fragments to be sequenced. Under the microwell layer is the hypersensitive ion sensor ISFET. All layers are contained within a semiconductor CMOS chip, similar to the chip used in the electronics industry. When dNTP is incorporated into a growing complementary chain, a hydrogen ion is released that excites a hypersensitive ion sensor. If homopolymer repeats are present in the sequence of the template, multiple dNTP molecules will be included in one cycle. This results in a corresponding amount of hydrogen atoms being released and in proportion to a higher electrical signal. This technology is different from other sequencing technologies that do not use modified nucleotides or optical devices. Additional details for Ion Torrent Technology are found in Science 327 (5970): 1190 (2010); US Patent Application Publication Nos. 20090026082, 20090127589, 20100301398, 20100197507, 20100188073, and 20100137143, each of which is incorporated by reference in its entirety.

In various embodiments, sequencing reads obtained from the NGS methods can be filtered by quality and grouped by barcode sequence using any algorithms known in the art, e.g., Python script barcodeCleanup.py. In some embodiments, a given sequencing read may be discarded if more than about 20% of its bases have a quality score (Q-score) less than Q20, indicating a base call accuracy of less than about 99%. In some embodiments, a given sequencing read may be discarded if more than about 5%, about 10%, about 15%, about 20%, about 25%, about 30% have a Q-score less than Q10, Q20, Q30, Q40, Q50, Q60, or more, indicating a base call accuracy of less than about 90%, less than about 99%, less than about 99.9%, less than about 99.99%, less than about 99.999%, less than about 99.9999%, or more, respectively.

In some embodiments, all sequencing reads associated with a barcode containing less than 50 reads may be discarded to ensure that all barcode groups, representing single cells, contain a sufficient number of high-quality reads. In some embodiments, all sequencing reads associated with a barcode containing less than 30, less than 40, less than 50, less than 60, less than 70, less than 80, less than 90, less than 100 or more reads may be discarded to ensure the quality of the barcode groups representing single cells.

Sequence reads with common barcode sequences (e.g., meaning that sequence reads originated from the same cell) may be aligned to a reference genome using known methods in the art to determine alignment position information. The alignment position information may indicate a beginning position and an end position of a region in the reference genome that corresponds to a beginning nucleotide base and end nucleotide base of a given sequence read. A region in the reference genome may be associated with a target gene or a segment of a gene. Example aligner algorithms include BWA, Bowtie, Spliced Transcripts Alignment to a Reference (STAR), Tophat, or HISAT2. Further details for aligning sequence reads to reference sequences are described in U.S. application Ser. No. 16/279,315, which is hereby incorporated by reference in its entirety. In various embodiments, an output file having SAM (sequence alignment map) format or BAM (binary alignment map) format may be generated and output for subsequent analysis.

### System and/or Computer Embodiments

Embodiments described herein further refer to example systems and computer embodiments for performing the variant calling methods described above. The subsequent description refers to the cell analysis workflow device 120 and base caller device 130, as described above in reference to FIG. 1.

In various embodiments, a cell analysis workflow device 120 includes at least a microfluidic device that is configured to encapsulate cells with reagents, encapsulate cell lysates with reaction mixtures, and perform nucleic acid amplification reactions. For example, the microfluidic device can include one or more fluidic channels that are fluidically connected. Therefore, the combining of an aqueous fluid through a first channel and a carrier fluid through a second channel results in the generation of emulsion droplets. In various embodiments, the fluidic channels of the microfluidic device may have at least one cross-sectional dimension on the order of a millimeter or smaller (e.g., less than or equal to about 1 millimeter). Additional details of microchannel design and dimensions are described in International Patent Application No. PCT/US2016/016444 and U.S. patent application Ser. No. 14/420,646, each of which is hereby incorporated by reference in its entirety. An example of a microfluidic device is the Tapestri™ Platform.

In various embodiments, the cell analysis workflow device 120 may also include one or more of: (a) a temperature control module for controlling the temperature of one or more portions of the subject devices and/or droplets therein and which is operably connected to the microfluidic device(s), (b) a detection module, i.e., a detector, e.g., an optical imager, operably connected to the microfluidic device(s), (c) an incubator, e.g., a cell incubator, operably connected to the microfluidic device(s), and (d) a sequencer operably connected to the microfluidic device(s). The one or more temperature and/or pressure control modules provides control over the temperature and/or pressure of a carrier fluid in one or more flow channels of a device. As an example, a temperature control module may be one or more thermal cycler that regulates the temperature for performing nucleic acid amplification. The one or more detection modules i.e., a detector, e.g., an optical imager, is configured for detecting the presence of one or more droplets, or one or more characteristics thereof, including their composition. In some embodiments, detection modules are configured to recognize one or more components of one or more droplets, in one or more flow channel. The sequencer is a hardware device configured to perform sequencing, such as next generation sequencing. Examples of sequencers include Illumina sequencers (e.g., MiniSeq™, MiSeq™, NextSeq™ 550 Series, or NextSeq™ 2000), Roche sequencing system 454, and Thermo Fisher Scientific sequencers (e.g., Ion GeneStudio S5 system, Ion Torrent Genexus System).

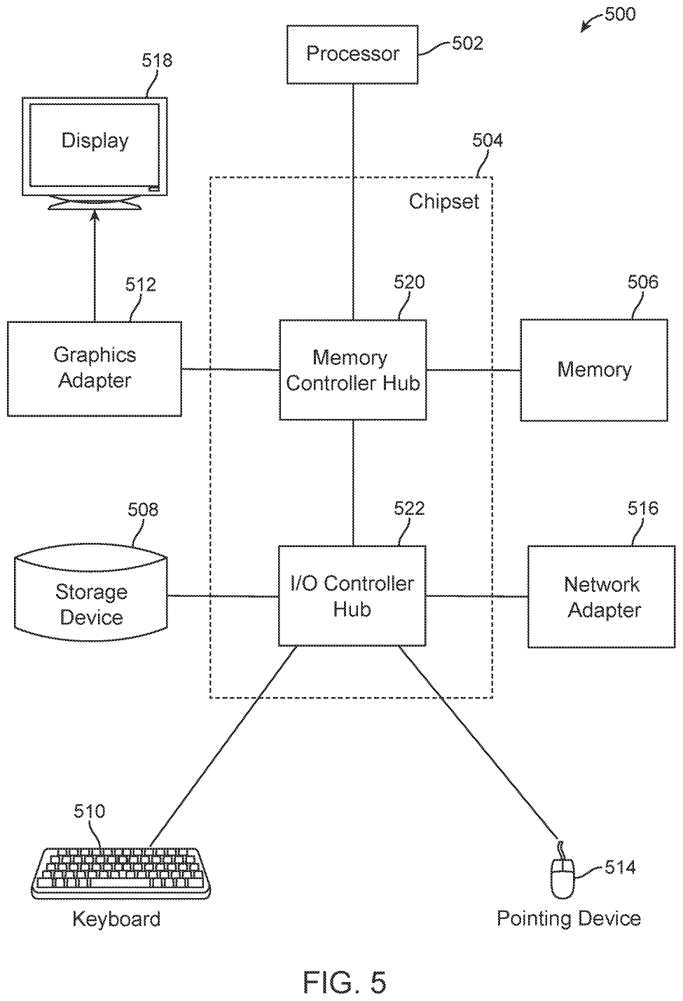

FIG. 5 depicts an example computing device for implementing system and methods described in reference to FIGS. 1-4. In various embodiments, the example computing device 500 serves as the base caller device 130 described in FIG. 1 for performing error corrections and for calling variants. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

As shown in FIG. 5, in some embodiments, the computing device 500 includes at least one processor 502 coupled to a chipset 504. The chipset 504 includes a memory controller hub 520 and an input/output (I/O) controller hub 522. A memory 506 and a graphics adapter 512 are coupled to the memory controller hub 520, and a display 518 is coupled to the graphics adapter 512. A storage device 508, an input interface 514, and network adapter 516 are coupled to the I/O controller hub 522. Other embodiments of the computing device 500 have different architectures.

The storage device 508 is a non-transitory computer-readable storage medium such as a hard drive, compact disk read-only memory (CD-ROM), DVD, or a solid-state memory device. The memory 506 holds instructions and data used by the processor 502. The input interface 514 is a touch-screen interface, a mouse, track ball, or other type of input interface, a keyboard, or some combination thereof, and is used to input data into the computing device 500. In some embodiments, the computing device 500 may be configured to receive input (e.g., commands) from the input interface 514 via gestures from the user. The graphics adapter 512 displays images and other information on the display 518. The network adapter 516 couples the computing device 500 to one or more computer networks.

The computing device 500 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 508, loaded into the memory 506, and executed by the processor 502.

The types of computing devices 500 can vary from the embodiments described herein. For example, the computing device 500 can lack some of the components described above, such as graphics adapters 512, input interface 514, and displays 518. In some embodiments, a computing device 500 can include a processor 502 for executing instructions stored on a memory 506.

The methods of performing base error corrections and variant calling can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of executing instructions for performing the base error corrections and variant calling methods disclosed herein. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

### Examples

### Example 1: Observed Base Errors in Sequence Reads Prior to Application of Error Correction Model

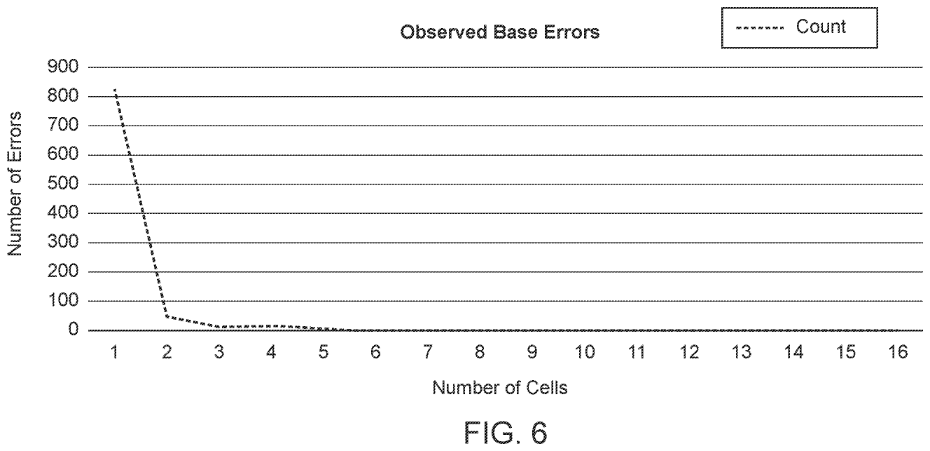

FIG. 6 depicts example a distribution of base errors, where a majority of base errors are observed in only one cell. The quantified errors in FIG. 6 refer to errors present in sequence reads without application of the error correction model.

The data were generated internally from cell line samples, run through Tapestri™ and analyzed using the Tapestri™ standard pipeline. The errors (mismatch) per cell were obtained and frequency of the errors in cells were computed to generate that plot. Specifically, a majority of errors in sequence reads are observed in only 1 cell, with a limited number of errors in sequence reads observed in more than 1 cell. This suggests that performing corrections on sequence reads in individual cells allows for reducing a number of errors (e.g., false positives and/or false negatives) that are mistakenly identified as a matched base or a mismatched base in relation to a reference base. In other words, if a base of a sequence read derived from a cell is determined to be an error, then it is more likely that the same base of other sequence reads derived from the same cell are errors. Performing a cell-specific error correction of sequence reads from individual cells thus is more accurate and/or faster than conventional methods (e.g., error correcting reads obtained through bulk processing).

### Example 2: Example Method of Implementing an Error Correction Model

Generally, the example methods for implementing an error correction model described below in relation to FIGS. 7-10 refer to performing error correction of bases in sequence reads derived from an individual cell.

A transition matrix, such as a transition matrix shown in FIG. 7, was generated for the sample. The probabilities of the transition matrix were generated by quantifying across known bases of 4 million reads of the sample, the reads aligned to a reference genome. For a known reference base (e.g., known reference base of adenine, thymine, guanine, or cytosine), the observed quantity of each of the 4 nucleotide bases across the 4 million probes was determined to generate the relative probabilities in the transition matrix.

Mismatched bases across sequence reads of cells were identified. For each base, a multinomial probability is calculated, the multinomial probability reflecting the likelihood of observing the proportion of alternate bases (e.g., any of 3 nucleotide bases that differs from the reference base) at a position across the sequence reads. In particular, the multinomial probability for a position was calculated according to:

P(Base 1=X,Base 2=Y,Base 3=Z|N reads)

where base 1, base 2, and base 3 refer to nucleotide bases that do not match with the reference base, where X is the number of observed sequence reads with base 1 at the position, where Y is the number of observed sequence reads with base 2 at the position, where Z is the number of observed sequence reads with base 3 at the position, and where N is the total number of observed sequence reads at the position.

The multinomial probabilities for bases were compared to transition probabilities of the transition matrix. Transition probabilities reflect the likelihood of transitioning from a reference nucleotide base to an observed nucleotide base. If the multinomial probability was greater than the transition probability of the transition matrix, the base was identified as a base of interest. If the multinomial probability was less than the transition probability of the transition matrix, the base was not identified as a base of interest.

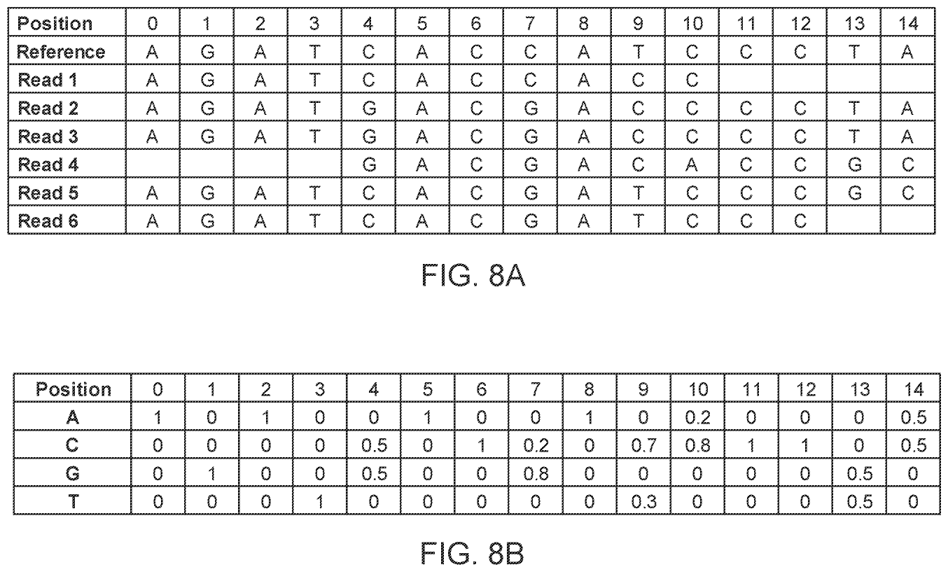

Pileups were created for each base of interest. FIGS. 8A and 8B are example depictions of a pileup of six sequence reads across different positions. FIGS. 8A and 8B each depict example positions 0-14 (top row). FIG. 8A further identifies the reference base at each of the corresponding positions (second row) as well as the bases of each of the six aligned sequence reads. FIG. 8B depicts the probabilities of each base that have been quantified across the six sequence reads. One skilled in the art can readily understand that additional positions across the genome (e.g., thousands or millions of positions), additional reference bases (e.g., thousands or millions of reference bases), and additional bases of sequence reads (e.g., thousands or millions of additional sequence reads) can be included in example pileups.

Here, the example pileup is generated for a base of interest that is mismatched in comparison to a reference base. In particular, the example pileup is generated for position 7. The reference base indicates a cytosine base at position 7, but five of the six sequence reads (83%) include a mismatched guanine base.

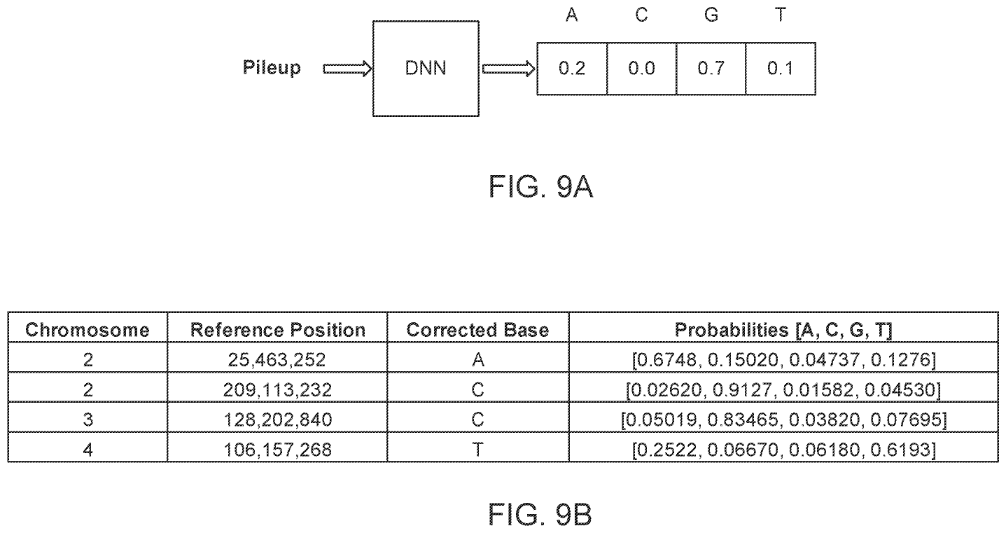

FIG. 9A depicts example input and output of the error correction model. In this example, the pileup, such as the pileup shown in FIG. 8B, is provided as input to the error correction model to correct a base of interest. Here, the error correction model is a deep learning neural network (DNN). The error correction model was optimized using several different hyper-parameters to identify the optimal values for each hyper-parameter. Hyper-parameters include but are not limited to kernel regularization coefficient, learning rate, number of layers, activation functions, and optimizers.

The error correction model analyzes single cell features of the pileup including contextual sequences around the base of interest, sequencing depth of the base of interest, and allele frequency of the base of interest.

The error correction model outputs a distribution of probabilities across the four nucleotide bases (adenine, cytosine, guanine, thymine), where each probability indicates the likelihood that the base of interest is a particular base. In the example shown in FIG. 9A, the error correction model outputs a distribution of probabilities indicating a likelihood of 20% that the base of interest is an adenine, a likelihood of 0% that the base of interest is a cytosine, a likelihood of 70% that the base of interest is a guanine, and a likelihood of 10% that the base of interest is a thymine.

FIG. 9B depicts an example of correcting bases of interest using probabilities predicted by the error correction model. The first two columns shown in FIG. 9B identify the location of the base, including the chromosome on which the base is located as well as the reference position of the base. The third column identifies the corrected base that the base of interest has been corrected to, which is dependent on the probabilities outputted by the error correction model. Here, the probabilities outputted by the error correction model are shown in the fourth column.

Specifically, for the first row, the outputted probabilities indicate that the base of interest is most likely an adenine nucleotide base, given that it has the highest probability (e.g., 0.6748). Thus, the base of interest is corrected to an adenine. For the second row, the outputted probabilities indicate that the base of interest is most likely a cytosine nucleotide base, given that it has the highest probability (e.g., 0.9127). For the third row, the outputted probabilities indicate that the base of interest is most likely a cytosine nucleotide base, given that it has the highest probability (e.g., 0.83465). For the fourth row, the outputted probabilities indicate that the base of interest is most likely a thymine nucleotide base, given that it has the highest probability (e.g., 0.6193).

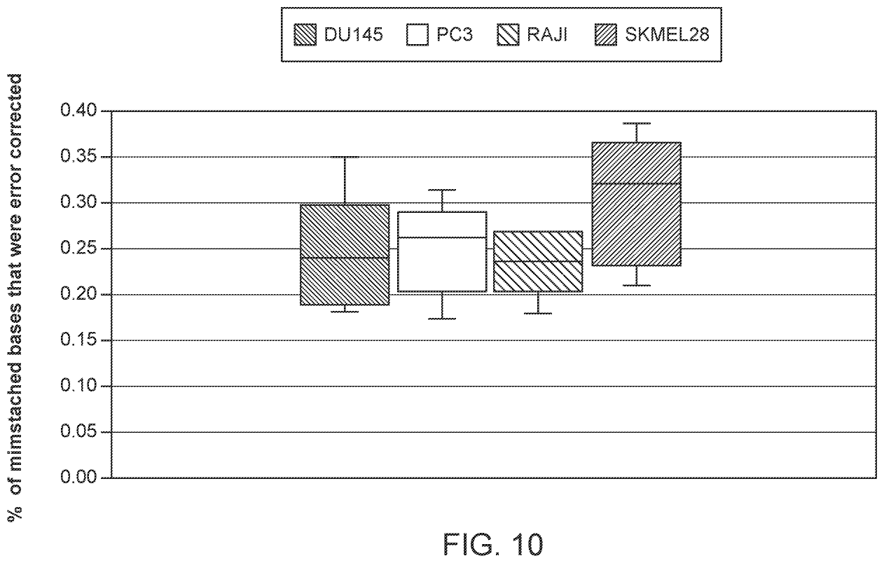

FIG. 10 demonstrates correction of 20-35% bases across four different cell populations as a result of implementing the error correction model. Each of the four cell lines were processed through a single cell workflow device (e.g., Tapestri®) and single cell DNA was sequenced to generate sequence reads. For each cell, the error correction model was applied to error correct bases of interest in sequence reads derived from the cell.

Altogether, the application of the error correction model to single cell DNA sequence reads can identify and correct a large proportion of erroneous bases which likely arise due to any of PCR errors, sequencing errors, sequencing alignment errors, or correction errors. These corrected sequence reads enables more accurate variant calls, as described below in Example 3.

### Example 3: Method of Implementing an Variant Caller Model

After error correcting the sequence reads, variants were filtered to remove variants that failed to meet thresholds such as minimum allele frequency and number of cells mutated. For each variant in the remaining variants cell population features were calculated: percentage of heterozygous calls, median variant allele frequency (VAF) of heterozygous calls, median genotype quality of heterozygous calls, median read depth of heterozygous calls, percentage of homozygous calls, median VAF of homozygous calls, median genotype quality of homozygous calls, median read depth of homozygous calls, percentage of reference calls, coefficient of variation (CV) of read depth for homozygous calls, CV of read depth for heterozygous calls, CV of genotype quality of homozygous calls, CV of genotype quality of heterozygous calls, CV of VAF for homozygous calls, CV of VAF for heterozygous calls, difference between mean and median VAF for homozygous calls, difference between mean and median VAF for heterozygous calls, and amplicon GC percentage.

Cell population features from cells obtained from 19 samples were used to train the variant caller model, which, in this scenario, was a multiclass neural network classifier. Training samples are disclosed below in Table 1. For these samples, the known variants were given classes (heterogeneous variant, homozygous variant, or reference base) based on known truth variants (confirmed from bulk sequencing methods) present in the respective samples. The training samples included diverse samples from cell mixtures at various dilutions up to 0.1% and clinical samples processed through Tapestri instrument and sequenced on a diverse set of sequencers. Since the training data had class imbalance where certain classes had far less calls compared to others, upsampling for smaller classes was performed. The hyper-parameters for the model were iteratively adjusted using validation data with known truth labels. Once the model achieved adequate accuracy training was stopped and then the model was used in prediction mode on new samples to identify top variants in those samples.

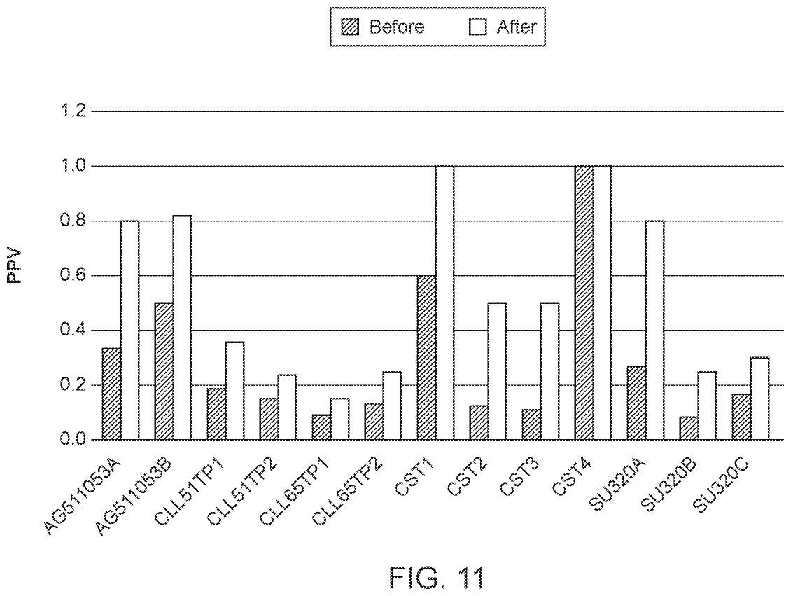

Thirteen test samples were used to evaluate the performance of the variant caller model. Test samples are disclosed below in Table 2. FIG. 11 demonstrates improved positive predictive value of true variants following implementation of the variant caller model across the 13 samples. With the two step error correction model and variant prediction model, a significantly improved median positive predictive value (PPV) was achieved. Specifically, a 2-3 fold improvement of PPV at 0.5% LOD was observed in a majority of the 13 samples. The 2-3 fold improvement was observed in comparison to a conventional GATK model which employs hard cutoff filters as opposed to the error correction model and/or variant prediction model.

TABLE 1
Training samples used to train the variant
caller model.

| | | | Number of |
|---|---|---|---|
| Panel | Sequencer | Sample | Samples |
| AML | MiSeq | Pure | 4 |
| AMLv2 | NextS eq | Titration | 4 |
| CLL | NovaSeq | Clinical | 3 |
| THP | MiSeq | Pure | 4 |
| AML | MiSeq | Titration | 4 |

| | |
|---|---|
| Total Samples | 19 |

TABLE 2
Test samples used to validate the variant
caller model.

| | | Sample | Number of |
|---|---|---|---|
| Panel | Sequencer | Name | Samples |
| AML | MiSeq/HiSeq | A | 4 |
| AML | HiSeq | B | 2 |
| CLL | HiSeq/NovaSeq | C | 4 |
| AML | HiSeq2500 | D | 3 |

| | |
|---|---|
| Total Samples | 13 |

Altogether, these results demonstrate that the application of the error correction model and variant caller model achieves a significant improvement in variant calling.

What is claimed is:

1. A method for calling one or more variants of a cell population, the method comprising:
obtaining a plurality of sequence reads from cells of the cell population;
for a plurality of cells in the cell population, correcting sequence reads obtained from the cell, the correction comprising:
identifying a base of interest of the sequence reads that differs from a reference base;
applying an error correction model to analyze single cell features of the base of interest, the error correction model trained to predict a probability for the base of interest; and
correcting the base of interest of the sequence reads derived from the cell;
generating cell population features by aggregating corrected sequence reads across cells of the cell population, the corrected sequence reads comprising corrected bases; and
applying a variant caller model to the cell population features derived from the aggregated sequence reads to identify one or more variants across the cell population.

2. The method of claim 1, wherein the single cell features comprise contextual sequences around the base of interest, sequencing depth of the base of interest, allele frequency of the base of interest, and allele frequency of bases in a window around the base of interest.

3. The method of claim 1 or 2, wherein identifying a base of interest of the sequence reads comprises applying a transition matrix comprising likelihoods of transition between reference bases and mismatched bases to a probability of observing a proportion of nucleotide bases across the sequence reads for a mismatched base.

4. The method of claim 3, wherein identifying a base of interest of the sequence reads further comprises:
determining the probability of observing a proportion of nucleotide bases across the sequence reads for the mismatched base; and
comparing the determined probability to a likelihood of transition from the transition matrix.

5. The method of claim 4, wherein responsive to the determined probability being greater than the likelihood of transition, identifying the mismatched base as a base of interest.

6. The method of claim 5, wherein the transition matrix is generated using training data comprising sequence reads derived from one or more sample populations of cells.

7. The method of claim 5, wherein the transition matrix is generated using the plurality of sequence reads from cells of the cell population.

8. The method of claim 5, wherein the likelihoods of transition in the transition matrix are dynamically updated as sequence reads of the one or more cells of the cell population are corrected.

9. The method of any one of claims 1-8, wherein the error correction model is a neural network.

10. The method of any one of claims 1-9, wherein the error correction model is a deep learning neural network comprising one or more layers that learn motifs and local sequence contexts around a base of interest.

11. The method of any one of claims 1-10, wherein correcting one or more sequence reads of the plurality of sequence reads derived from the cell results comprises correcting at least 25% of bases of interest that differ from reference bases.

12. The method of any one of claims 1-11, wherein the cell population features comprise one or more of percentage of heterozygous calls, median variant allele frequency (VAF) of heterozygous calls, median genotype quality of heterozygous calls, median read depth of heterozygous calls, percentage of homozygous calls, median VAF of homozygous calls, median genotype quality of homozygous calls, median read depth of homozygous calls, percentage of reference calls, coefficient of variation (CV) of read depth for homozygous calls, CV of read depth for heterozygous calls, CV of genotype quality of homozygous calls, CV of genotype quality of heterozygous calls, CV of VAF for homozygous calls, CV of VAF for heterozygous calls, difference between mean and median VAF for homozygous calls, difference between mean and median VAF for heterozygous calls, and amplicon GC percentage.

13. The method of any one of claims 1-12, wherein the variant caller model predicts at least one of a heterozygous variant of interest or a homozygous variant of interest.

14. The method of claim 13, wherein the variant caller model further predicts indeterminate variants.

15. The method of any one of claims 1-14, wherein the variant caller model is trained using training data comprising sequence reads derived from one or more cell lines and indications of known heterozygous or homozygous variants present in the one or more cell lines.

16. The method of any one of claims 1-15, wherein the application of the error correction model and the variant caller model achieves at least a two-fold increase in true variant positive predictive value at a limit of detection (LOD) of 0.5% in comparison to a conventional GTAK variant caller.

17. The method of any one of claims 1-15, wherein the application of the error correction model and the variant caller model achieves a true variant positive predictive value of at least 0.6 at a limit of detection (LOD) of 0.5%.

18. The method of any one of claims 1-17, wherein the plurality of sequence reads derived from the cell are determined through a single-cell workflow analysis.

19. The method of any one of claims 1-18, wherein the reference base is determined from a reference genome sequence.

20. The method of any one of claims 1-18, wherein the reference base is determined from one or more sequence reads obtained from a control cell.

21. A non-transitory computer readable medium for calling one or more variants of a cell population, the non-transitory computer readable medium comprising instructions that, when executed by a processor, cause the processor to:
obtain a plurality of sequence reads from cells of the cell population;
for a plurality of cells in the cell population, correcting sequence reads obtained from the cell, the correction comprising:
identifying a base of interest of the sequence reads that differs from a reference base;
applying an error correction model to analyze single cell features of the base of interest, the error correction model trained to predict a probability for the base of interest;
correcting the base of interest of the sequence reads derived from the cell;
generating cell population features by aggregating corrected sequence reads across cells of the cell population, the corrected sequence reads comprising corrected bases; and
applying a variant caller model to the cell population features derived from the aggregated sequence reads to identify one or more variants across the cell population.

22. The non-transitory computer readable medium of claim 21, wherein the single cell features comprise contextual sequences around the base of interest, sequencing depth of the base of interest, allele frequency of the base of interest, and allele frequency of bases in a window around the base of interest.

23. The non-transitory computer readable medium of claim 21 or 22, wherein the instructions that cause the processor to identify a base of interest of the sequence reads further comprises instructions that, when executed by the processor, cause the processor to apply a transition matrix comprising likelihoods of transition between reference bases and mismatched bases.

24. The non-transitory computer readable medium of claim 23, wherein the instructions that cause the processor to identify a base of interest of the sequence reads further comprises instructions that, when executed by the processor, cause the processor to:
determine a probability of observing proportion of nucleotide bases across the sequence reads for a mismatched base; and
compare the determined probability to a likelihood of transition from the transition matrix.

25. The non-transitory computer readable medium of claim 24, wherein responsive to the determined probability being greater than the likelihood of transition, identifying the mismatched base as a base of interest.

26. The non-transitory computer readable medium of any one of claims 23-25, wherein the transition matrix is generated using training data comprising sequence reads derived from one or more sample populations of cells.

27. The non-transitory computer readable medium of any one of claims 23-25, wherein the transition matrix is generated using the plurality of sequence reads from cells of the cell population.

28. The non-transitory computer readable medium of any one of claims 23-25, wherein the likelihoods of transition in the transition matrix are dynamically updated as sequence reads of the one or more cells of the cell population are corrected.

29. The non-transitory computer readable medium of any one of claims 21-28, wherein the error correction model is a neural network.

30. The non-transitory computer readable medium of any one of claims 21-29, wherein the error correction model is a deep learning neural network comprising one or more layers that learn motifs and local sequence contexts around a base of interest.

31. The non-transitory computer readable medium of any one of claims 21-30, wherein correcting one or more sequence reads of the plurality of sequence reads derived from the cell results comprises correcting at least 25% of bases of interest that differ from a reference base.

32. The non-transitory computer readable medium of any one of claims 21-31, wherein the cell population features comprise one or more of percentage of heterozygous calls, median variant allele frequency (VAF) of heterozygous calls, median genotype quality of heterozygous calls, median read depth of heterozygous calls, percentage of homozygous calls, median VAF of homozygous calls, median genotype quality of homozygous calls, median read depth of homozygous calls, percentage of reference calls, coefficient of variation (CV) of read depth for homozygous calls, CV of read depth for heterozygous calls, CV of genotype quality of homozygous calls, CV of genotype quality of heterozygous calls, CV of VAF for homozygous calls, CV of VAF for heterozygous calls, difference between mean and median VAF for homozygous calls, difference between mean and median VAF for heterozygous calls, and amplicon GC percentage.

33. The non-transitory computer readable medium of any one of claims 21-32, wherein the variant caller model predicts at least one of a heterozygous variant of interest or a homozygous variant of interest.

34. The non-transitory computer readable medium of claim 33, wherein the variant caller model further predicts indeterminate variants.

35. The non-transitory computer readable medium of any one of claims 21-34, wherein the variant caller model is trained using training data comprising sequence reads derived from one or more cell lines and indications of known heterozygous or homozygous variants present in the one or more cell lines.

36. The non-transitory computer readable medium of any one of claims 21-35, wherein the application of the error correction model and the variant caller model achieves at least a two-fold increase in true variant positive predictive value at a limit of detection (LOD) of 0.5% in comparison to a conventional GTAK variant caller.

37. The non-transitory computer readable medium of any one of claims 21-35, wherein the application of the error correction model and the variant caller model achieves a true variant positive predictive value of at least 0.6 at a limit of detection (LOD) of 0.5%.

38. The non-transitory computer readable medium of any one of claims 21-37, wherein the plurality of sequence reads derived from the cell are determined through a single-cell workflow analysis.

39. The non-transitory computer readable medium of any one of claims 21-38, wherein the reference base is determined from a reference genome sequence.

40. The non-transitory computer readable medium of any one of claims 21-38, wherein the reference base is determined from one or more sequence reads obtained from a control cell.

41. A system comprising:
a single-cell analysis workflow device configured to generate a plurality of sequence reads for cells in a cell population;
a computational device communicatively coupled to the single-cell analysis workflow device, the computational device configured to:
obtain a plurality of sequence reads from cells of the cell population;
for a plurality of cells in the cell population, correcting sequence reads obtained from the cell, the correction comprising:
identifying a base of interest of the sequence reads that differs from a reference base;
applying an error correction model to analyze single cell features of the base of interest, the error correction model trained to predict a probability for the base of interest;
correcting the base of interest of the sequence reads derived from the cell;
generating cell population features by aggregating corrected sequence reads across cells of the cell population, the corrected sequence reads comprising corrected bases; and
applying a variant caller model to the cell population features derived from the aggregated sequence reads to identify one or more variants across the cell population.

42. The system of claim 41, wherein the single cell features comprise contextual sequences around the base of interest, sequencing depth of the base of interest, allele frequency of the base of interest, and allele frequency of bases in a window around the base of interest.

43. The system of claim 41 or 42, wherein identifying a base of interest of the sequence reads comprises: applying a transition matrix comprising likelihoods of transition between reference bases and mismatched bases to a probability of observing a proportion of nucleotide bases across the sequence reads for a mismatched base.

44. The system of claim 43, wherein identifying a base of interest of the sequence reads comprises:
determining the probability of observing proportion of nucleotide bases across the sequence reads for the mismatched base; and
comparing the determined probability to a likelihood of transition from the transition matrix.

45. The system of claim 44, wherein responsive to the determined probability being greater than the likelihood of transition, identifying the mismatched base as a base of interest.

46. The system of claim 45, wherein the transition matrix is generated using training data comprising sequence reads derived from one or more sample populations of cells.

47. The system of claim 45, wherein the transition matrix is generated using the plurality of sequence reads from cells of the cell population.

48. The system of claim 45, wherein the likelihoods of transition in the transition matrix are dynamically updated as sequence reads of the one or more cells of the cell population are corrected.

49. The system of any one of claims 41-48, wherein the error correction model is a neural network.

50. The system of any one of claims 41-49, wherein the error correction model is a deep learning neural network comprising one or more layers that learn motifs and local sequence contexts around a base of interest.

51. The system of any one of claims 41-50, wherein correcting one or more sequence reads of the plurality of sequence reads derived from the cell results comprises correcting at least 25% of bases of interest that differ from a reference base.

52. The system of any one of claims 41-51, wherein the cell population features comprise one or more of percentage of heterozygous calls, median variant allele frequency (VAF) of heterozygous calls, median genotype quality of heterozygous calls, median read depth of heterozygous calls, percentage of homozygous calls, median VAF of homozygous calls, median genotype quality of homozygous calls, median read depth of homozygous calls, percentage of reference calls, coefficient of variation (CV) of read depth for homozygous calls, CV of read depth for heterozygous calls, CV of genotype quality of homozygous calls, CV of genotype quality of heterozygous calls, CV of VAF for homozygous calls, CV of VAF for heterozygous calls, difference between mean and median VAF for homozygous calls, difference between mean and median VAF for heterozygous calls, and amplicon GC percentage.

53. The system of any one of claims 41-52, wherein the variant caller model predicts at least one of a heterozygous variant of interest or a homozygous variant of interest.

54. The system of claim 53, wherein the variant caller model further predicts indeterminate variants.

55. The system of any one of claims 41-54, wherein the variant caller model is trained using training data comprising sequence reads derived from one or more cell lines and indications of known heterozygous or homozygous variants present in the one or more cell lines.

56. The system of any one of claims 41-55, wherein the application of the error correction model and the variant caller model achieves at least a two-fold increase in true variant positive predictive value at a limit of detection (LOD) of 0.5% in comparison to a conventional GTAK variant caller.

57. The system of any one of claims 41-55, wherein the application of the error correction model and the variant caller model achieves a true variant positive predictive value of at least 0.6 at a limit of detection (LOD) of 0.5%.

58. The system of any one of claims 41-57, wherein the reference base is determined from a reference genome sequence.

59. The system of any one of claims 41-57, wherein the reference base is determined from one or more sequence reads obtained from a control cell.
